(19) United States
(12) Patent Application Publication
(10) Pub. No.: US 2013/0274772 A1
(43) Pub. Date: Oct. 17, 2013

(54) APPARATUSES AND METHODS FOR ANASTOMOSIS

(71) Applicant: Children's National Medical Center, Washington, DC (US)

(72) Inventors: Peter C. W. Kim, Washington, DC (US); Axel Krieger, Alexandria, VA (US); Katherine Davenport, San Diego, CA (US); Alana Beres, Montreal (CA); Carolyn Cochenour, Washington, DC (US); Justin Opfermann, Washington, DC (US)

(21) Appl. No.: 13/864,156

(22) Filed: Apr. 16, 2013

Related U.S. Application Data
(60) Provisional application No. 61/624,690, filed on Apr. 16, 2012.

(51) Int. Cl. A61B 17/11

(52) U.S. Cl. CPC A61B 17/11
USPC 606/153

(57) ABSTRACT

Apparatuses and methods for performing anastomosis of distal and proximal vessels. An exemplary apparatus includes a distal anastomosis includes a distal hub and a distal engagement segment. The distal hub is configured to detachably connect to a distal catheter. The distal engagement segment is connected to the distal hub and includes one or more distal arm segments that are configured to engage an inner wall of the distal vessel.

### CROSS-REFERENCE TO RELATED APPLICATION

This application is related to and claims priority under 35 U.S.C. §119(e) to U.S. patent application No. 61/624,690, filed Apr. 16, 2012, which is incorporated herein by reference in its entirety.

### BACKGROUND OF THE INVENTION

1. Field of the Invention

Embodiments described herein relate generally to apparatuses and methods for performing an anastomosis.

2. Background

Anastomosis refers to the connection of two structures, such as blood vessels or other body vessels. Typical devices and methods for anastomosis require access to the body vessels for joining through a surgical opening (e.g., laparoscopic port, thoracoscopic port, full surgical opening).

### SUMMARY OF THE INVENTION

The present embodiments are directed to anastomosis structures, catheter deployment systems, and methods for using the same to reduce the invasiveness of anastomosis procedures.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a simplified anatomical drawing of esophageal atresia with fistula closed.

FIGS. 2a-d illustrates an overview of deploying an expandable anastomosis structure to reconnect an esophagus.

FIG. 3a illustrates an exemplary expandable anastomosis structure deployed in an esophagus.

FIG. 3b illustrates an exemplary umbrella anastomosis structure deployed in an esophagus.

FIG. 4 is a more detailed view of an embodiment of an expandable anastomosis structure deployed in an esophagus with connecting barbed suture and an expandable biodegradable impermeable membrane.

FIG. 5 is a more detailed view of a variation of an expandable anastomosis structure: an hourglass expandable anastomosis structure deployed in an esophagus with connecting barbed suture and an expandable biodegradable impermeable membrane.

FIG. 6 is a more detailed view of another variation of an expandable anastomosis structure: a spiral expandable anastomosis structure deployed in an esophagus with connecting barbed suture and an expandable biodegradable impermeable membrane.

FIG. 7 is a cross sectional view of a center ring of an exemplary anastomosis structure showing how a barbed suture moves through a channel and thus locking the anastomosis structure in the expanded configuration.

FIG. 8 is an exemplary top view of a deployed anastomosis structure, showing an example of how a barbed suture feeds through both proximal and distal ends of an anastomosis structure.

FIGS. 9a-f illustrate a method for deploying an exemplary expandable anastomosis structure.

FIG. 10 illustrates a detailed view of an embodiment using an hourglass-shaped umbrella anastomosis structure deployed in an esophagus with a connecting barbed suture.

FIG. 11 illustrates a detailed view of a variation of an hourglass-shaped umbrella anastomosis structure with a rigid distal umbrella deployed in an esophagus with a connecting barbed suture.

FIG. 12 illustrates a detailed view of another variation of an umbrella anastomosis structure using a flexible material for arms deployed in an esophagus with a connecting barbed suture.

FIG. 13 illustrates a detailed view of another variation of an umbrella anastomosis structure, using living hinges to collapse the structure and allow for opening when unsheathed, deployed in an esophagus with a connecting barbed suture.

FIGS. 14a-d illustrate a method for deploying an exemplary hourglass-shaped umbrella anastomosis structure.

FIGS. 15a-f illustrate a method for deploying an exemplary hourglass-shaped umbrella anastomosis structure with a rigid distal umbrella.

FIG. 16 illustrates a cross-section of an exemplary deployment catheter system showing a threaded interaction between proximal and distal umbrella anastomosis structures, respectively.

FIG. 17 illustrates a motion of turning a handle of an exemplary deployment catheter system to unscrew and release an anastomosis structure.

FIG. 18 illustrates an embodiment of a release mechanism for an anastomosis structure, where inner catheters flex inward by pinching a handle to release proximal and distal ends of an anastomosis structure.

FIG. 19 illustrates an embodiment of a release mechanism for an anastomosis structure in a cross-sectional view, showing a notch interaction between the anastomosis structure and deployment catheter system, as well as a motion of internal spring actuators.

### DETAILED DESCRIPTION

According to one embodiment of the present disclosure, there is described an apparatus for joining portions of a body vessel. The apparatus includes a catheter, having a distal catheter portion, and a distal umbrella structure. The distal umbrella structure has a distal hinge ring, a distal umbrella actuation mechanism, and a distal umbrella. The distal umbrella includes a plurality of mechanically connected spokes with barbs disposed thereon. The barbs are configured to pierce and hook tissue.

According to another embodiment of the apparatus, the distal umbrella actuation mechanism includes a concentric shaft configured to engage with the distal umbrella through a wedge mechanism. The engagement results in expansion of the distal umbrella.

According to another embodiment of the apparatus, the distal hinge ring and the distal umbrella are detachable from the distal catheter.

According to another embodiment of the apparatus, the distal hinge ring and the distal umbrella are hollow.

According to another embodiment of the apparatus, the distal hinge ring and the distal umbrella are biodegradable.

According to another embodiment of the apparatus, the distal umbrella further includes a plurality of U-shaped clips configured to provide a retention force on the vessel.

According to another embodiment of the apparatus, the apparatus further includes an endoscope.

According to another embodiment of the apparatus, the apparatus further includes a proximal catheter portion and a proximal umbrella structure. The proximal umbrella structure includes a proximal hinge ring, a proximal umbrella actuation mechanism, and a proximal umbrella. The proximal umbrella includes a plurality of mechanically connected spokes with barbs disposed thereon. The barbs are configured to pierce and hook tissue. Further, the distal catheter portion and the proximal catheter portion are concentric.

According to another embodiment of the apparatus, the proximal umbrella actuation mechanism includes a concentric shaft configured to engage with the proximal umbrella through a cam mechanism. The engagement results in expansion of the proximal umbrella.

According to another embodiment of the apparatus, the proximal hinge ring and the proximal umbrella are detachable from the proximal catheter.

According to another embodiment of the apparatus, the proximal hinge ring and the proximal umbrella are hollow.

According to another embodiment of the apparatus, the proximal hinge ring and the proximal umbrella are biodegradable.

According to another embodiment of the apparatus, the proximal umbrella further includes a plurality of U-shaped clips configured to provide a retention force on the vessel.

According to another embodiment of the apparatus, the apparatus further includes a mechanical snapping mechanism configured to lock the distal umbrella and the proximal umbrella in expanded positions.

According to another embodiment of the apparatus, the apparatus is configured such that the spokes of the distal umbrella are evenly spaced from the spokes of the proximal umbrella when the distal umbrella and the proximal umbrella are in expanded positions.

According to one embodiment of the present disclosure, there is provided a method for joining portions of a body vessel. A distal catheter portion is inserted into a distal portion of the vessel. The distal catheter portion includes a distal umbrella comprising a plurality of mechanically connected spokes with barbs disposed thereon. The distal umbrella is actuated such that the distal umbrella expands, and the barbs pierce, hook, and open the distal portion of the vessel. The distal umbrella is locked such that the distal umbrella provides a radial force on the vessel. The distal umbrella is detached from the distal catheter portion. Further, the distal catheter portion is removed from the vessel.

According to another embodiment of the method, the method further includes inserting a proximal catheter portion into a proximal portion of the vessel. The proximal catheter portion includes a proximal umbrella comprising a plurality of mechanically connected spokes with barbs disposed thereon. The proximal umbrella is actuated such that the proximal umbrella expands, and the barbs pierce, hook, and open the proximal portion of the vessel. The distal umbrella and the proximal umbrella are approximated. The proximal umbrella is locked such that the proximal umbrella provides a radial force on the vessel. The proximal umbrella is detached from the proximal catheter portion. The proximal catheter portion is removed from the vessel.

According to another embodiment of the method, the method further includes facilitating the advancement of the distal catheter portion into the distal vessel with a guide wire.

According to one embodiment of the present disclosure, there is provided an apparatus for performing anastomosis of distal and proximal vessels. The apparatus includes a distal anastomosis section. The distal anastomosis section includes a distal hub and a distal engagement segment. The distal hub is configured to detachably connect to a distal catheter. The distal engagement segment is connected to the distal hub and includes one or more distal arm segments that are configured to engage an inner wall of the distal vessel.

According to another embodiment of the apparatus, the distal hub is hollow.

According to another embodiment of the apparatus, the apparatus further includes the distal catheter. The distal catheter includes one or more distal graspers that are configured to engage the inner wall of the distal vessel. The distal catheter is configured to pull the distal vessel towards the proximal vessel while the one or more distal graspers engage the inner wall of the distal vessel.

According to another embodiment of the apparatus, the one or more distal graspers contain Nitinol.

According to another embodiment of the apparatus, the apparatus further includes a proximal anastomosis section. The proximal anastomosis section includes a proximal hub and a proximal engagement segment. The proximal hub is configured to detachably connect to a proximal catheter. The proximal engagement segment is connected to the proximal hub and includes one or more proximal arm segments that are configured to engage the proximal vessel.

According to another embodiment of the apparatus, the proximal hub is hollow.

According to another embodiment of the apparatus, the distal anastomosis section and the proximal anastomosis section are part of a single anastomosis structure. Further,

the distal engagement segment is connected to the proximal engagement segment.

According to another embodiment of the apparatus, the single anastomosis structure is an hourglass shaped structure.

According to another embodiment of the apparatus, one or a combination of the one or more distal arm segments and the one or more proximal arm segments contain living hinges.

According to another embodiment of the apparatus, the apparatus further includes a fluid impermeable membrane configured to prevent fluid leakage through the distal and proximal engagement segments.

According to another embodiment of the apparatus, the apparatus further includes the distal catheter and the proximal catheter. The distal catheter includes one or more distal graspers that are configured to engage the inner wall of the distal vessel. The distal catheter is configured to pull the distal vessel towards the proximal vessel while the one or more distal graspers engage the inner wall of the distal vessel. The proximal catheter includes one or more proximal graspers that are configured to engage the inner wall of the proximal vessel to stabilize the proximal vessel while the distal vessel is being pulled towards the proximal vessel.

According to another embodiment of the apparatus, one or a combination of the one or more distal and proximal graspers contain Nitinol.

According to another embodiment of the apparatus, the distal engagement segment is configured to engage the distal vessel and the proximal engagement segment is configured to engage the proximal vessel when the distal and proximal catheters are moved towards each other.

According to another embodiment of the apparatus, the distal and proximal anastomosis sections are separate anastomosis structures.

According to another embodiment of the apparatus, the distal anastomosis section further includes a distal deployment mechanism configured to deploy the distal engagement segment such that the one or more distal arm segments engage the inner wall of the distal vessel.

According to another embodiment of the apparatus, the distal deployment mechanism includes a sheath that compresses the one or more distal arm segments against the distal catheter. Further, the one or more distal arm segments are configured to deploy when the sheath is removed from the one or more distal arm segments.

According to another embodiment of the apparatus, the apparatus further including the distal catheter. The distal catheter is configured to pull the distal vessel towards the proximal vessel while the one or more distal arm segments engage the inner wall of the distal vessel.

According to another embodiment of the apparatus, the distal anastomosis section is a rigid structure.

According to another embodiment of the apparatus, one or a combination of the distal and proximal anastomosis sections is a flexible structure that is configured to expand radially away from the distal catheter.

According to another embodiment of the apparatus, the apparatus includes the distal catheter. The distal catheter includes one or more distal graspers that are configured to engage the inner wall of the distal vessel. The distal catheter is configured to pull the distal vessel towards the proximal vessel while the one or more distal graspers engage the inner wall of the distal vessel.

According to another embodiment of the apparatus, the proximal anastomosis section further includes a proximal deployment mechanism configured to deploy the proximal engagement segment such that the one or more proximal arm segments engage the inner wall of the proximal vessel.

According to another embodiment of the apparatus, a connector configured to couple the distal engagement segment to the proximal engagement segment.

According to another embodiment of the apparatus, the connector is a barbed suture.

According to one embodiment of the present disclosure, there is provided a method for performing anastomosis of distal and proximal vessels. A distal catheter is inserted through a proximal vessel and into a distal vessel. The distal catheter has detachably mounted thereon a distal anastomosis section, including a distal hub and a distal engagement segment. The distal hub is configured to detachably connect to the distal catheter. The distal engagement segment is connected to the distal hub and includes one or more distal arm segments that are configured to engage an inner wall of the distal vessel.

According to another embodiment of the method, the method further includes inserting the distal catheter through the proximal vessel via a natural orifice.

According to another embodiment of the method, the method further includes deploying one or more distal graspers that are connected to the distal catheter and configured to engage the inner wall of the distal vessel. Further, The distal catheter is retracted to pull the distal vessel towards the proximal vessel while the one or more distal graspers engage the inner wall of the distal vessel.

According to another embodiment of the method, the method further includes inserting a proximal catheter through the proximal vessel. The proximal catheter has detachably mounted thereon a proximal anastomosis section, including a proximal hub and a proximal engagement segment. The proximal hub is configured to detachably connect to the proximal catheter. The proximal engagement segment is connected to the proximal hub and includes one more proximal arm segments that are configured to engage the proximal vessel.

According to another embodiment of the method, the distal anastomosis section and the proximal anastomosis section are part of a single anastomosis structure. Further, the distal engagement segment is connected to the proximal engagement segment.

According to another embodiment of the method, the method further includes deploying one or more distal graspers that are connected to the distal catheter and configured to engage the inner wall of the distal vessel. One or more proximal graspers that are connected to the proximal catheter and configured to engage the inner wall of the proximal vessel is deployed to stabilize the proximal vessel while the distal vessel is being pulled towards the proximal vessel. The distal catheter is retracted to pull the distal vessel towards the proximal vessel while the one or more distal graspers engage the inner wall of the distal vessel.

According to another embodiment of the method, the method further includes moving the distal and proximal catheters towards each other such that the distal engagement segment engages the distal vessel and the proximal engagement segment engages the proximal vessel.

According to another embodiment of the method, the distal and proximal anastomosis sections are separate anastomosis structures.

According to another embodiment of the method, the method further includes actuating a distal deployment mechanism, included in the distal anastomosis section, to deploy the distal engagement segment such that the one or more distal arm segments engage the inner wall of the distal vessel.

According to another embodiment of the method, the method further includes retracting the distal catheter to pull the distal vessel towards the proximal vessel while the one or more distal arm segments engage the inner wall of the distal vessel.

According to another embodiment of the method, the distal anastomosis section is a rigid structure.

According to another embodiment of the method, the method further includes deploying one or more distal graspers that are connected to the distal catheter and configured to engage the inner wall of the distal vessel. Further, the distal catheter is retracted to pull the distal vessel towards the proximal vessel while the one or more distal graspers engage the inner wall of the distal vessel.

According to another embodiment of the method, the method further includes actuating a proximal deployment mechanism, included in the proximal anastomosis section, to deploy the proximal engagement segment such that the one or more proximal arm segments engage the inner wall of the proximal vessel.

While the present disclosure is susceptible of embodiment in many different forms, there is shown in the drawings and will herein be described in detail specific embodiments, with the understanding that the present description of such embodiments is to be considered as an example of the principles and not intended to limit the present disclosure to the specific embodiments shown and described. In the description below, like reference numerals are used to describe the same, similar or corresponding parts in the several views of the drawings.

Embodiments of the present disclosure are directed to apparatuses and associated methods for performing joining of body vessels (anastomosis). The embodiments provide a unique and simple way of deploying expandable structures to engage two vessels and then join and secure the vessels to form the anastomosis. In certain embodiments, the expandable structures may be made of biodegradable materials which are absorbed by the body after wound closure, thus eliminating potential complications associated with implanted materials (e.g., infections, occlusions) or the need for an invasive removal procedure.

According to embodiments of the present disclosure, an anastomosis can be performed through a natural orifice. Example applications for a natural orifice anastomosis device (NOAD) and related method include esophageal atresia repair and urethra repair after radical prostatectomy. Thus, anastomosis may be performed without the need of a surgical opening by accessing the site of the anastomosis through a natural body orifice (e.g., mouth, anus, urinary meatus) with a catheter delivery system.

NOAD provides a surgeon with a catheter based minimally invasive method by which to access the site for the anastomosis through a natural body orifice and reduce the associated trauma of the procedure. NOAD differs from other catheter devices and methods for anastomosis at least by allowing anastomosis through the deployment of an expandable or umbrella anastomosis structure, with multiple arms containing engagement regions with barbs, hooks, or microstructures to join and secure body vessels.

In certain embodiments, the structure is collapsed within a deployment catheter system. Before deployment, the deployment catheter system is advanced through a natural orifice to a most distal body vessel. The distal body vessel is engaged by deploying an expandable grasping structure fixed to the deployment catheter system or a distal portion of an umbrella anastomosis structure. The distal body vessel is then brought into the proximal body vessel by actuating the deployment catheter system. An expandable structure or proximal portion of an umbrella anastomosis structure is deployed and engages with a proximal vessel wall. Depending on the embodiment, the proximal portion may engage with the distal portion of the umbrella anastomosis structure. The expandable or umbrella anastomosis structure is secured in the deployed state sealing the anastomosis through radial pressure and longitudinal tension. The expandable or umbrella anastomosis structure is then released from the deployment catheter system and the deployment catheter system is removed. The expandable or umbrella anastomosis structure remains at the anastomosis site, maintaining the anastomosis until the wound is healed.

The expandable and/or umbrella anastomosis structures can be manufactured from biodegradable materials or combinations of biodegradable and non-biodegradable materials. The biodegradable material is absorbed by the body after the wound is healed and may be comprised of, but is not limited to the following materials: Polyglycolic acid (PGA), poly lactic-co-glycolic acid (PLGA), Poly-lactic acid (PLA), poly-L-lactide (PLLA), collagen, Magnesium Alloys, Iron based alloys, or any combination of the previously mentioned materials. Depending on the embodiment, the non-biodegradable sections may be such that they can pass through the exit bodily lumen without harm, such as the gastrointestinal tract. Non-biodegradable materials that may be used include, but are not limited to: stainless steel, Nitinol, colbalt alloys, and platinum alloys. The listed materials can exist in multiple forms such as films or extrusions. The anastomosis structures may be manufactured through laser cutting extrusions, injection molding of the parts, micromachining or 3D printing.

The implanted structure(s) may or may not cross the lumen, but allow the adequate passage of air and fluids, thus not occluding the body vessel after anastomosis. NOAD can be deployed through a single natural body orifice or by using an additional access site into the second body vessel. Before clinical acceptance of NOAD can be reached, it is conceivable that a small port for visual and tactile aid is used in addition to the access through the body orifice.

The implanted device, in certain embodiments, will have markers to be visible using a variety of post-implant monitoring modalities such as MR, ultrasound, and fluoroscopy. This will allow for non-invasive monitoring of the implant position and degradation over time.

Embodiments of the present disclosure, however, are not limited to applications requiring access through one or more natural orifices. In other embodiments, the anastomosis device and related method may require access through one or more surgical openings, such as in the case of coronary graft anastomosis for a coronary bypass procedure.

Congenital atresia of the esophagus describes a variety of malformations of esophageal continuity, and can occur with or without a fistula (abnormal connection) to the trachea (Hollwarth, M E. Pediatric Surgery: Diagnosis and Management, Ch33. 2009).

Nationwide, rates range between 1.04 and 4.91 per 10,000 live births (National Birth Defects Prevention Network 2006). Surgical correction of the anomaly involves closure of the fistula if present, and reestablishment of esophageal continuity. In order to achieve this, the procedure is traditionally performed through an incision in the chest, and the two ends of the esophagus are stitched together. This portion of the procedure is both technically demanding and time consuming. Those with advanced laparoscopic skills can perform this procedure in a minimally invasive manner, but the esophageal anastomosis remains the critical portion of the surgery. Complications from the procedure can include a leak at the anastomotic site, which presents early, or a stricture, which would present later.

Radical Prostatectomy is a well-established surgical procedure to remove all parts of the prostate gland, typically performed for patients with localized prostatic carcinoma. Since the prostate gland surrounds the urethra, the procedure requires division and subsequent re-attachment of urethral stump to bladder neck. 156,000 radical prostatectomy cases requiring urethra repair were performed in 2007 (National Hospital Discharge Survey 2007).

Other examples of procedures for the embodiments of the present disclosure include cystectomy (bladder removal) with ileal conduit creation, reattachment of ureters to the native bladder (ureteral reimplant/neocystotomy), and continent diversions (Indiana pouch, Mainz pouch, Miami pouch, neobladders) where ureter is connected to the bowel or any gastrointestinal (GI) segment for urinary diversion. Example procedures also include attachment of the ureter from donor kidneys into native bladders for transplant surgeries, ureteral re-anastomosis (e.g. after removal of a diseased segment) where the two ends of the ureter are reattached or one end of one ureter is attached to the opposite ureter, and anastomosis of fallopian tubes for reversal of tubal ligations.

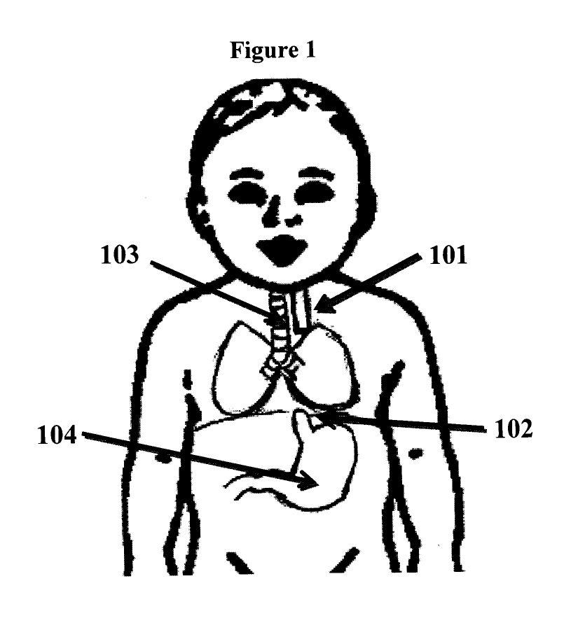

As described above, embodiments of the present disclosure generally involve apparatuses and methods for performing an anastomosis for example in esophageal atresia repairs. However, it should be noted that the embodiments are also applicable to performing anastomosis on any one or a combination of body vessels, or other body or artificial conduits. Referring to FIG. 1 a proximal (101) and distal (102) portion of a body vessel (e.g., esophagus) requiring joining is shown. FIG. 1 also shows the relevant anatomy surrounding the atresia including the trachea (103) and stomach (104).

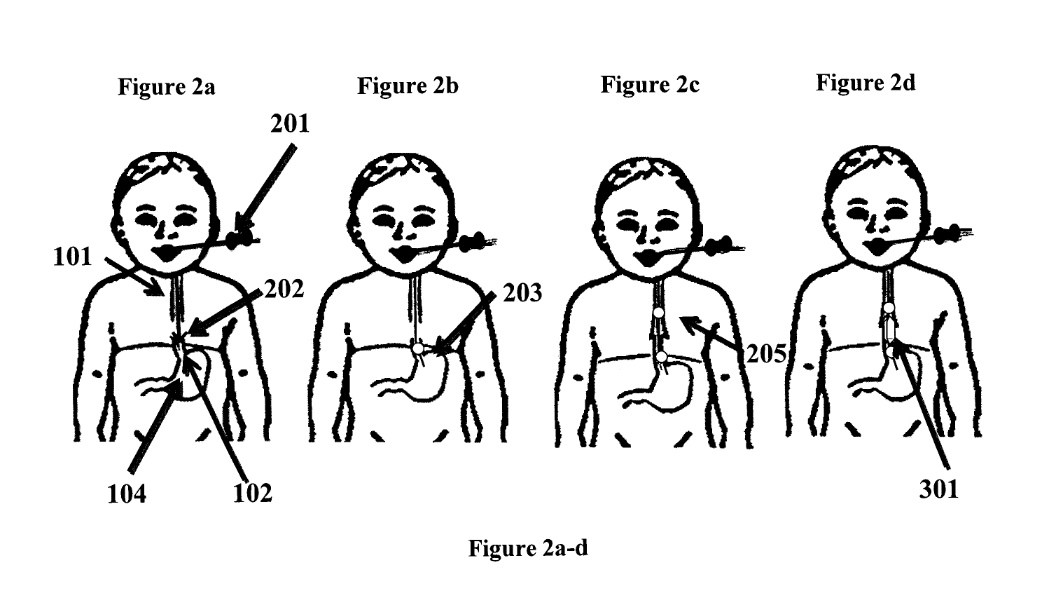

FIGS. 2a-d illustrates a generalization of performing an anastomosis using a catheter deployment system (201). Although, the disclosure refers to a child it should be understood that the anastomosis may be performed on persons of any age, or other animals. FIG. 2a illustrates a child with an esophageal atresia with a catheter deployment system (201) to grasp the distal esophagus (102) using an umbrella structure having distal graspers (202). FIG. 2b illustrates a child with an esophageal atresia with the catheter deployment system (201) with an inflated distal grasper balloon (203) and expanded graspers (202) to grasp the distal portion of the body vessel (102, e.g., the esophagus). FIG. 2c illustrates a child with the proximal (101) and distal (102) portions of the body vessel stabilized by proximal graspers (205). FIG. 2d illustrates a child with the deployment of an anastomosis structure (301) to join the proximal (101) and distal (102) portions of the body vessel (e.g., esophageal ends).

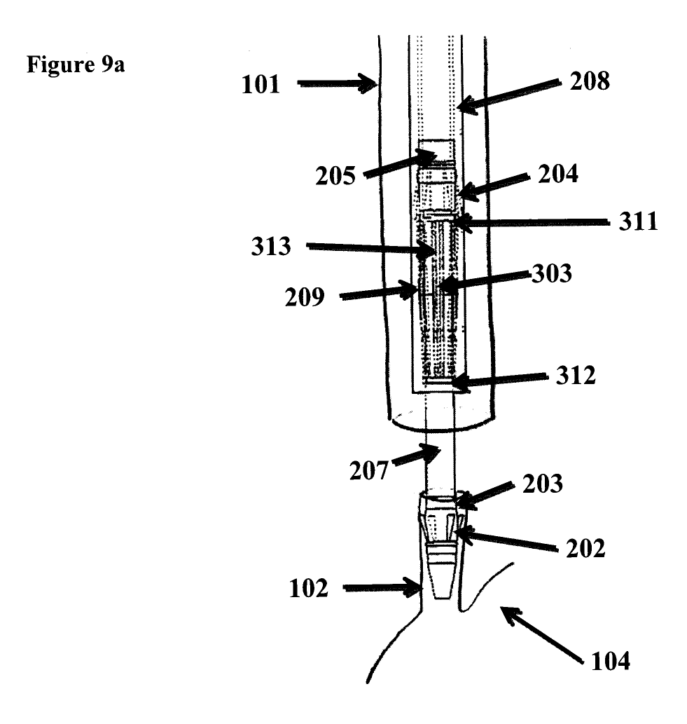

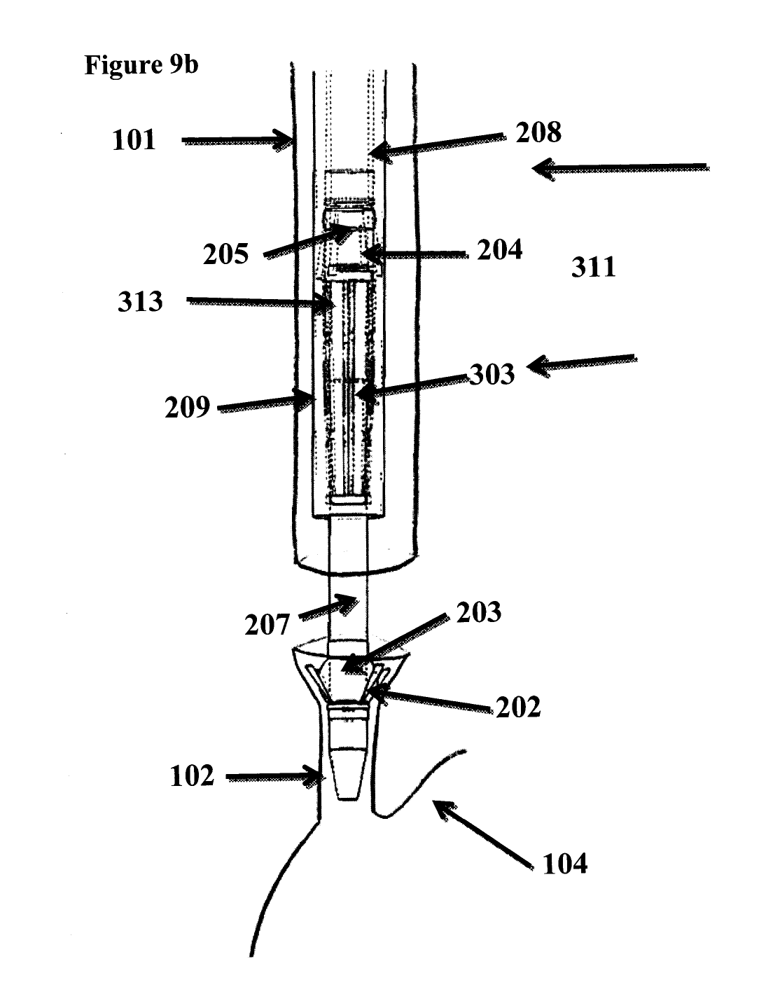

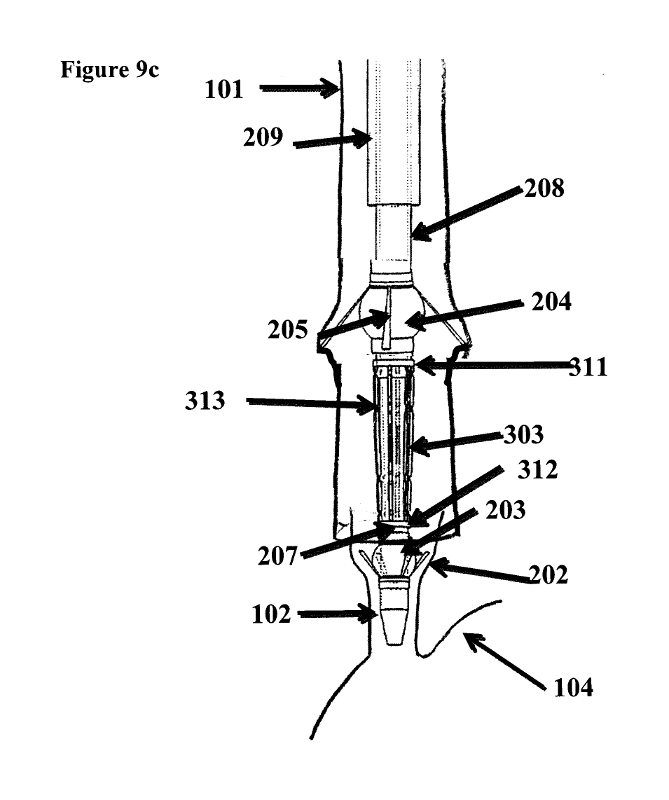

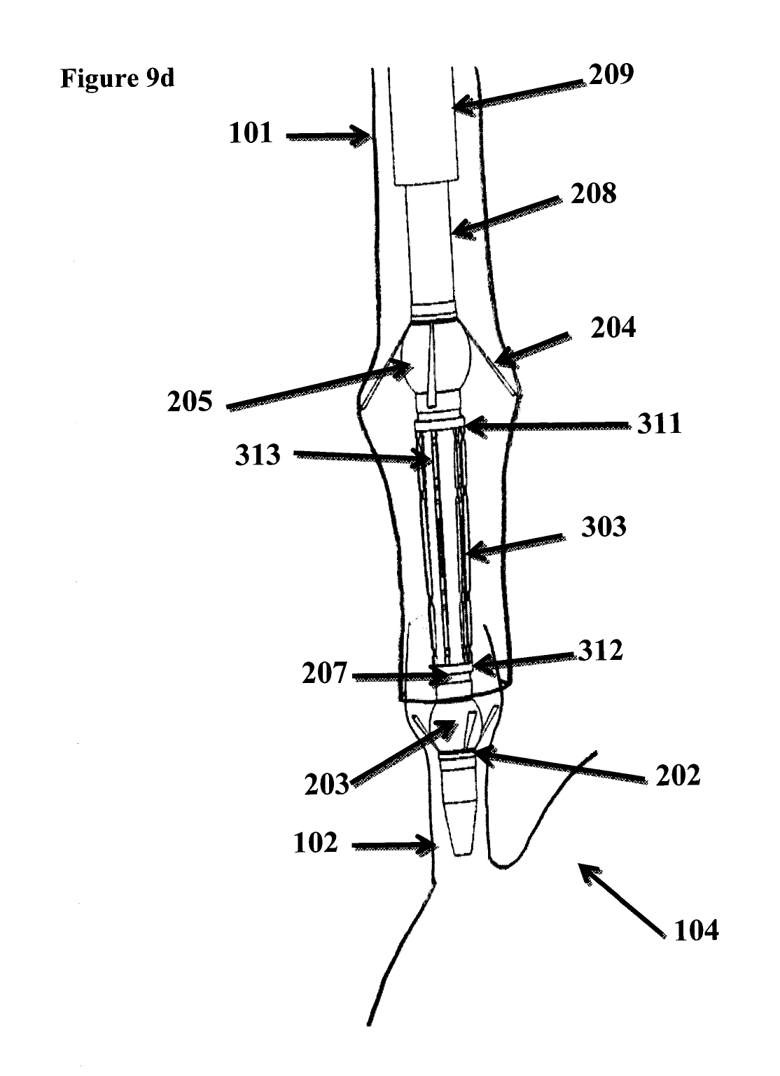

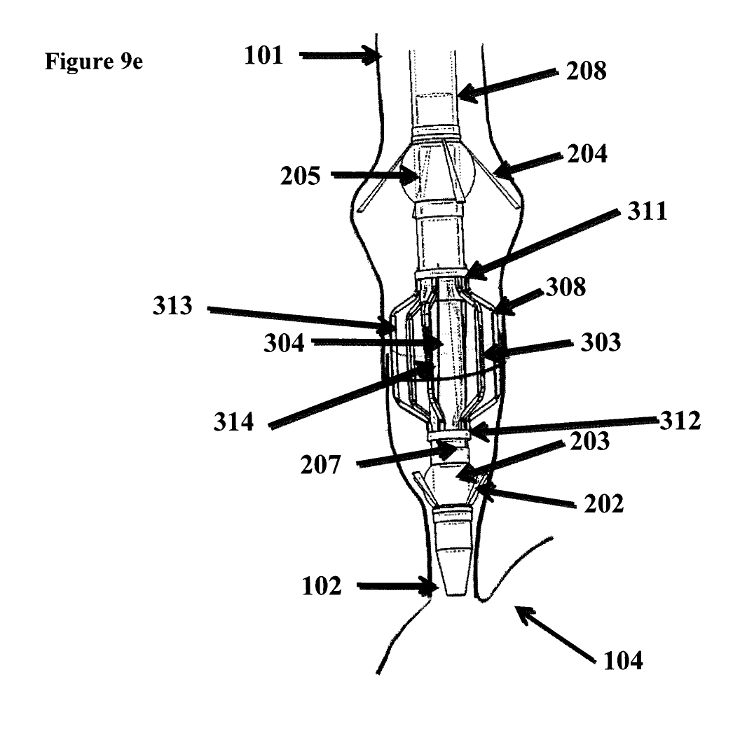

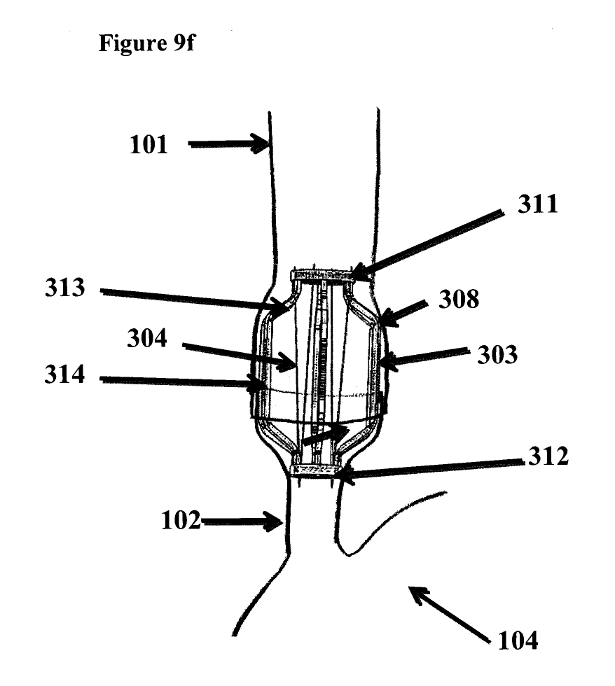

According to one embodiment of the present disclosure, a catheter such as the catheter illustrated in FIG. 9a, including a concentric inner catheter of a distal anastomosis device (207) and an inner catheter of proximal anastomosis device (208) is inserted through a natural orifice (e.g., mouth) and advanced to the site of the desired anastomosis. In case the proximal vessel (101) is closed (e.g., esophageal fistula), the inner catheter of the distal anastomosis device (207) is used to penetrate through the proximal vessel (101).

While esophageal atresia is shown as the example in many of the drawings, this catheter deployment system (201) may be used to join any two body vessels accessed through a natural orifice.

Expandable Anastomosis Structure

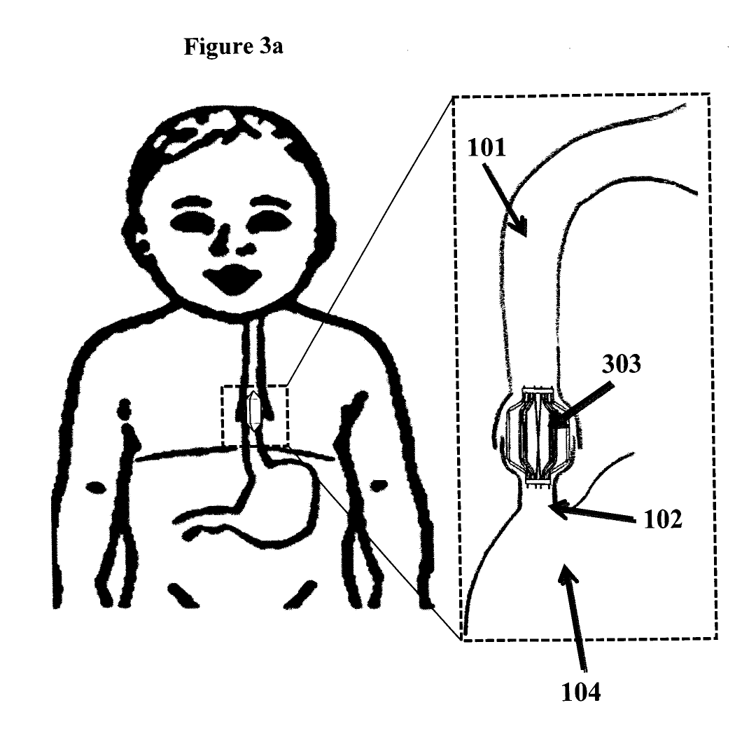

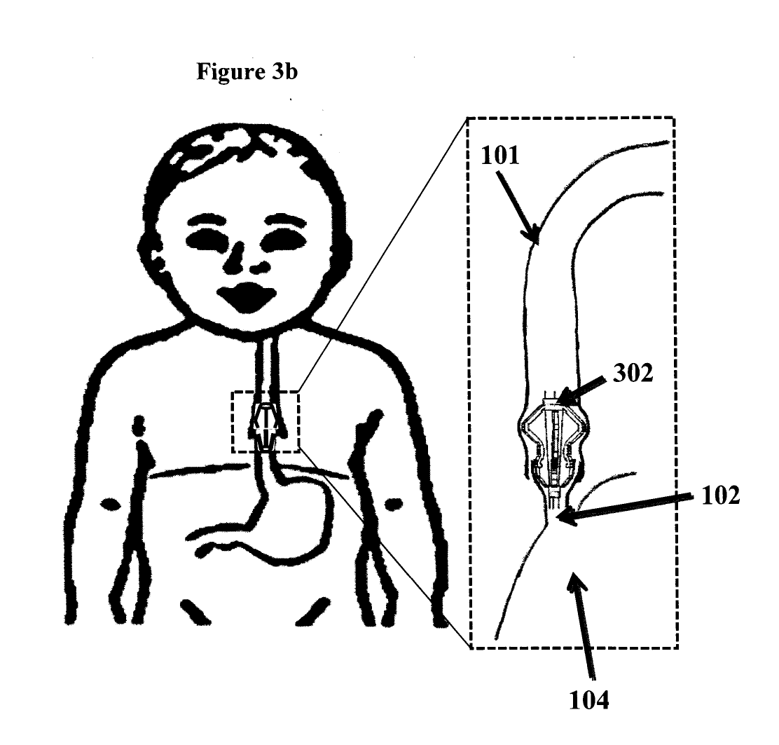

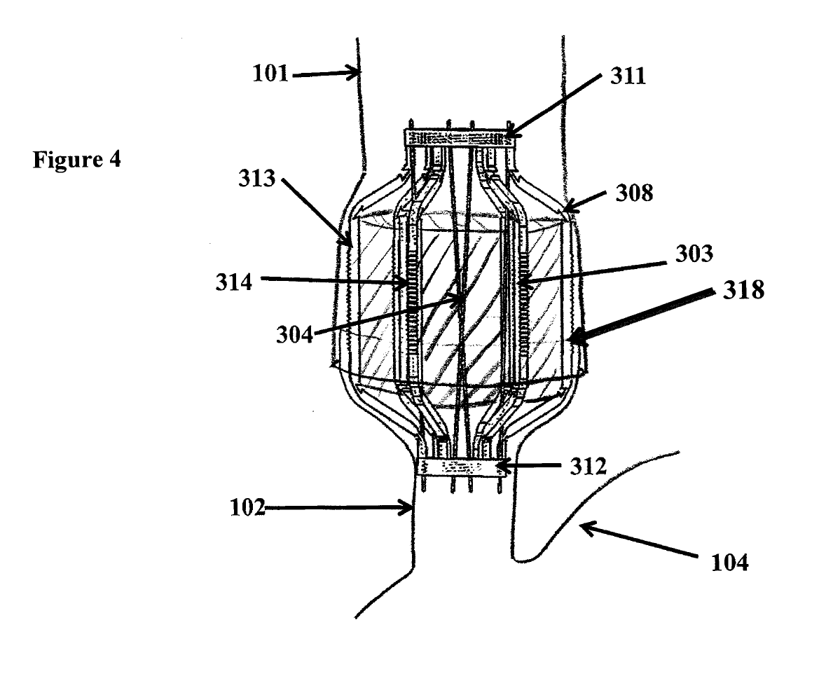

There are two categories of the anastomosis structure 301 shown in FIGS. 3a-b. The first category is an expandable anastomosis structure (303). FIG. 4 illustrates an example of the expandable anastomosis structure (303), which includes a concentric proximal ring (311) and distal ring (312) that ride over the deployment catheter system (201) and are connected by arms (313) that expand when the two rings (311, 312) are brought closer together by the actuating catheters in the deployment catheter system (201). There can be any number of arms depending on the two bodily vessels being brought together; FIG. 4 shows an example with 6 arms (313).

As shown in FIG. 4, the arms may flex due to living hinges (308) in the design of a rigid material or by the use of a flexible material. Barbed sutures (304) run through the concentric proximal ring (311) and distal (312) ring to keep the expandable anastomosis structure (303) in the expanded state. The arms may contain barbs (314), hooks or a microtexture that will engage and secure the expandable anastomosis device (303) to the proximal vessel (101) and distal vessel (102) walls by, for example, piercing and hooking tissue.

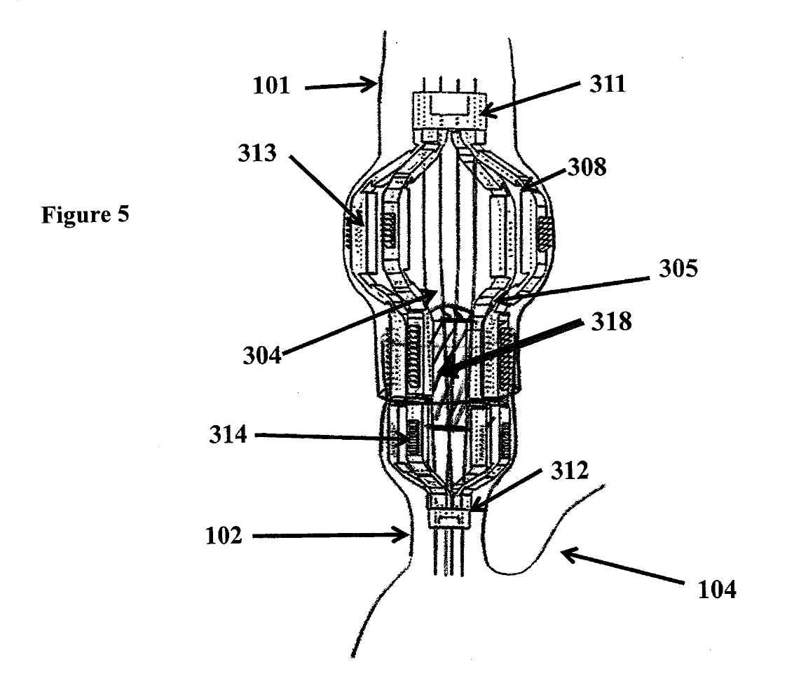

Referring to FIG. 4, the arms may contain only four living hinges (308) to give the structure a cage-like appearance. In addition, there may be an expandable biodegradable membrane (318) stretching between the arms (313) to create an impermeable seal to saliva, or other liquids. Other variations of the expandable anastomosis structure (303) include arms with multiple living hinges (308) that contour to the different sizes of the proximal vessel (101) and distal vessel (102) lumens shown in FIG. 5 as the hourglass-shaped expandable anastomosis structure (305). This variation can have any number of arms depending on the size of the two bodily vessels being brought together.

FIG. 5 shows an example with 6 arms (313). The arms (313) may contain barbs (314), hooks or a microtexture that will engage and secure the hourglass-shaped expandable anastomosis device (305) to the proximal vessel (101) and distal (102) vessel walls. In addition there may be an expandable biodegradable membrane (318) stretching between the arms to create an impermeable seal to saliva, or other liquids.

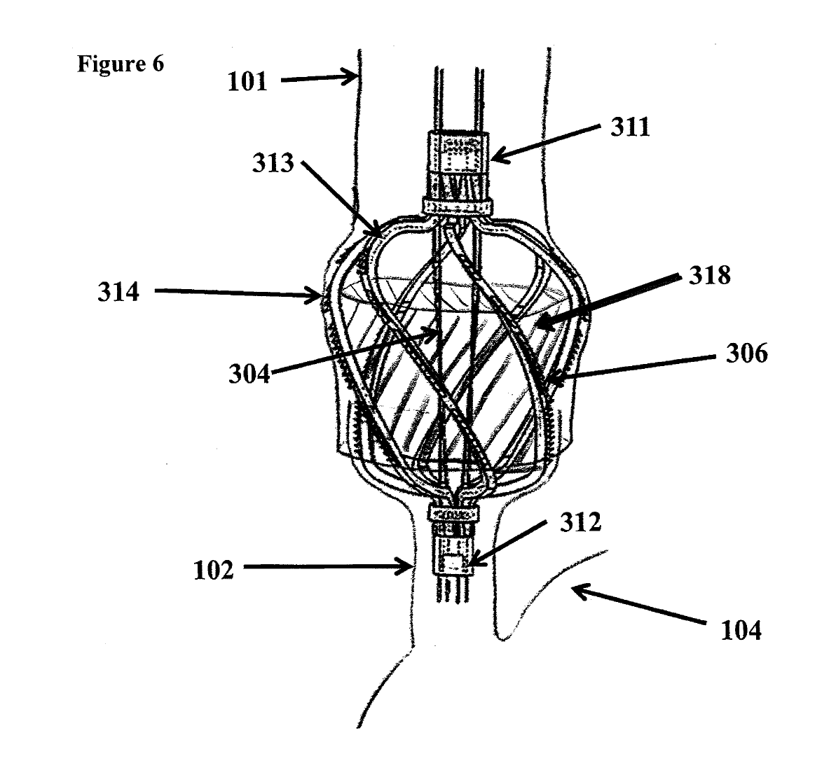

Yet another variation of the expandable anastomosis structure is shown in FIG. 6 as a spiral expandable anastomosis structure (306). This variation contains flexible arms (313) cut in a spiral fashion that expand and engage the proximal vessel (101) and distal vessel (102) walls in a spiral pattern. The arms may contain barbs (314), hooks or a microtexture that will engage and secure the spiral expandable anastomosis device (306) to the proximal vessel (101) and distal vessel (102) walls. This variation can have any number of arms depending on the size of the two bodily vessels being brought together;

FIG. 6 shows an example with 6 arms (313). In addition there may be an expandable biodegradable membrane (318) stretching between the arms (313) to create an impermeable seal to saliva, or other liquids. Additionally the spiral expandable anastomosis structure (306) may be straight along the length of the body or taper.

All three variations shown in FIGS. 4-6; expandable anastomosis structure (303), hourglass expandable anastomosis structure (305), and spiral expandable anastomosis structure (306) are shown with barbed suture (304) running through the concentric proximal ring (311) and distal ring (312) that secure the expandable anastomosis structures (303, 305, 306) in the expanded state. All three variations; expandable anastomosis structure (303), hourglass-shaped expandable anastomosis structure (305), and spiral expandable anastomosis structure (306) may be constructed of biodegradable materials or a combination of biodegradable and non-biodegradable materials such as but not limited to: biodegradable-(Polyglycolic acid (PGA), poly lactic-co-glycolic acid (PLGA), Poly-lactic acid (PLA), poly-L-lactide (PLLA), collagen, Magnesium Alloys, Iron based alloys, or any combination of the previously mentioned materials) or non-biodegradable-(stainless steel, Nitinol, colbalt alloys, and platinum alloys). Biodegradable materials allow for the anastomosis structure to be used more specifically in children without halting growth of the child resulting in strictures later in life.

Deployment and Catheter System for Expandable Anastomosis Structure

FIGS. 9a-f shows an embodiment of the deployment of the expandable anastomosis structure (303). The expandable anastomosis structure (303) is delivered to the atresia over the concentric inner catheter of the proximal anastomosis device (208) and inner catheter of the distal portion of the catheter deployment system (207) in the collapsed state as seen in FIG. 9a. The distal end of the distal portion of the catheter deployment system (207) is advanced through a natural orifice (e.g., the mouth) into the distal body vessel (102) (e.g., esophagus).

With the distal graspers (202) and distal grasper balloon (203) fully in the distal portion of the vessel (102), FIG. 9b shows the inflation of the distal graspers (202) to engage the distal vessel (102) wall. The distal graspers (202) are made from Nitinol or a material of equivalent properties (necessary properties include a high degree of elasticity and/or shape memory properties) and may contain any number of arms; FIGS. 9a-e show four armed graspers. The distal graspers (202) lay flat on the inner catheter of the distal portion of the deployment catheter system (207) when the grasper balloon (203) is deflated, as illustrated in FIG. 9a.

The distal graspers (202) are attached to the inner catheter of the distal of the catheter deployment system (207) just below the distal grasper balloon (203) so that they can flex outward without permanent deformation and then again lay flat when the distal grasper balloon (203) is deflated. The inner catheter of the distal of the catheter deployment system (207) can move independently of the inner catheter of the proximal portion of the catheter deployment system (208) and sheath (209).

FIG. 9c shows the proximal grasper balloon (204) inflated to expand the proximal graspers (205) to engage the proximal vessel (101) wall to stabilize the body vessel (e.g., the esophagus). The proximal graspers (205) are made from Nitinol or a material of equivalent properties and may contain any number of arms; FIGS. 9a-e show four armed graspers.

With the distal vessel wall (102) engaged by the expanded distal graspers (202) and proximal graspers (204), FIG. 9d shows the distal vessel (102) being pulled into the proximal vessel (101) by the moving the inner catheter of the distal portion of the catheter deployment system (207) and the distal graspers (202) independently of the inner catheter of the proximal portion of the catheter deployment system (208) so that there is an overlapping of the proximal vessel (101) and distal vessel (102) ends. Now both the proximal (101) and distal (102) vessels are overlapped and stabilized with the proximal graspers (204) and distal graspers (202) so that the deployment of the expandable anastomosis device (303) is reliable. By overlapping the proximal vessel (101) and distal vessel (102) the risk of leakage from the anastomosis site is minimized. However, in other embodiments, the proximal vessel (101) and distal vessel (102) need not overlap.

FIG. 9e shows the deployment of the expandable anastomosis structure (303) by actuating concentric catheters (207, 208) to expand the anastomosis structure (303) and secure it to the body vessel wall (e.g., esophageal wall). In one embodiment, the expandable anastomosis structure (303) are actuated through pulling together the inner catheter of the distal portion of the catheter deployment system (207) and the inner catheter of the proximal portion of the catheter deployment system (208) to cause the expandable anastomosis structure (303) to bend in a reliable manner at the site of the living hinges (308). This bending expands the expandable anastomosis structure (303) outward radially from the proximal ring (311) and distal ring (312), and the barbs (314) hook into the tissue sealing the anastomosis site. Each arm (313) remains in contact with the proximal vessel (101), distal vessel (102) and the overlapping regions to maintain an outward radial force on the vessels.

Barbed Suture

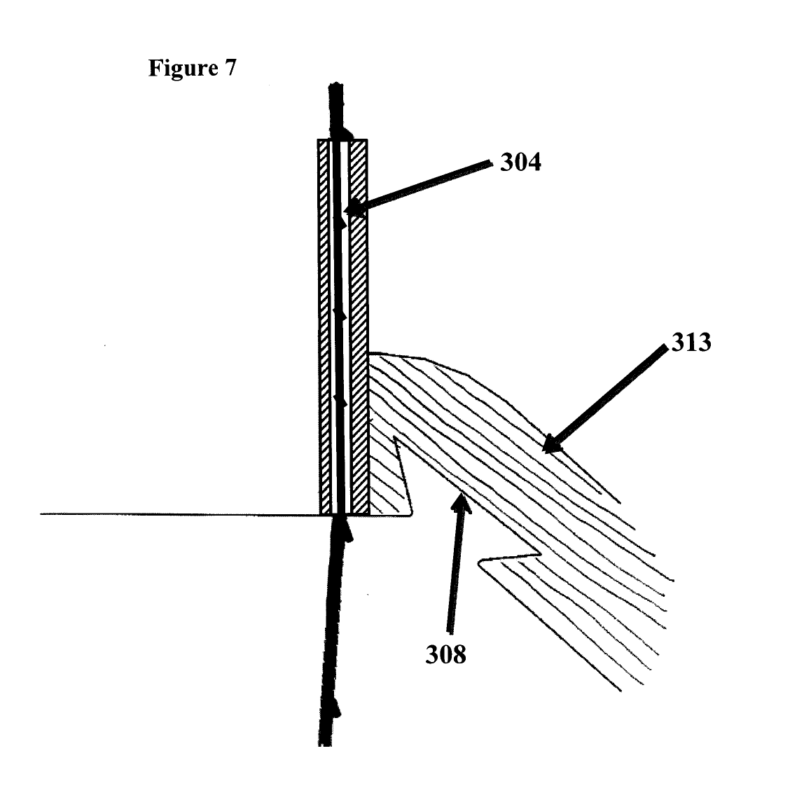

As the inner catheter of the proximal portion of the catheter deployment system (208) and inner catheter of the distal portion of the catheter deployment system (207) are actuating thus expanding the expandable anastomosis structure (303), as illustrated in FIG. 9e, there are barbed sutures (304) passing through straight channels in the proximal ring (311) shown in FIG. 7 and maintaining a fixed position on the distal ring (312). FIG. 7 shows that the barbs (304) are angled in such a way that the suture (304) can pass up through the proximal ring (311) but not back through, so that the proximal ring (311) and distal ring (312) can move closer to each other but not further away. This keeps the expandable anastomosis structure (303) in the deployed state providing the longitudinal tension to keep the proximal vessel (101) and distal (102) vessels together.

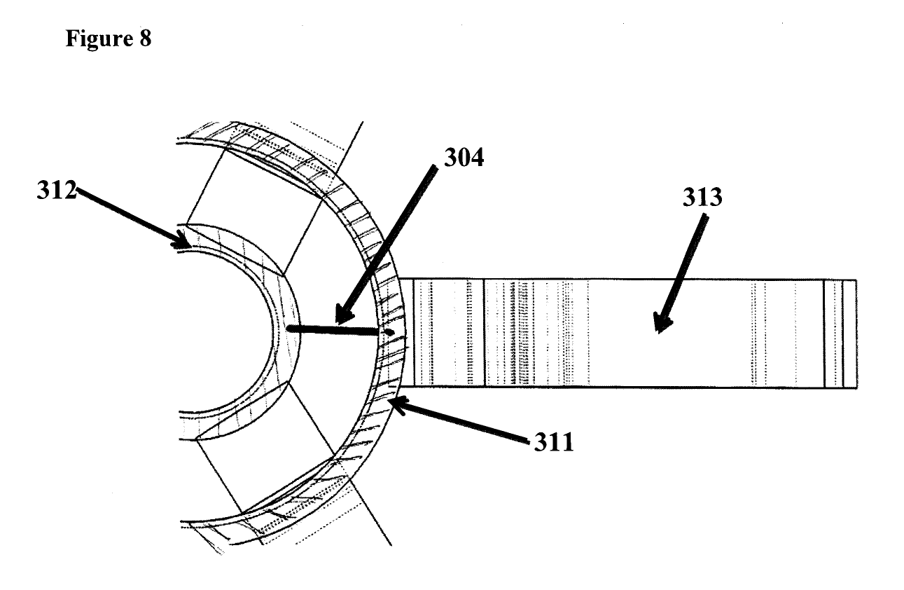

FIG. 8 shows a top view of the expandable anastomosis structure (303) with the barbed suture (304) passing through the top view of the proximal ring (309) and the top view of the distal ring (310). Depending on the embodiment, there may be one or more sutures running between the proximal (311) and distal (312) rings. Additionally, the one or more barbed suture (304) may be made of biodegradable materials.

While barbed sutures (304) may be used to create the longitudinal tension between proximal and distal ends of the anastomosis structure (303), there are a number of mechanisms that may serve the same function such as but not limited to: mechanical locking features, clips to tie off the ends of the suture, a snap fit lock, or a material transition change such as the hardening of epoxy.

Screw Release

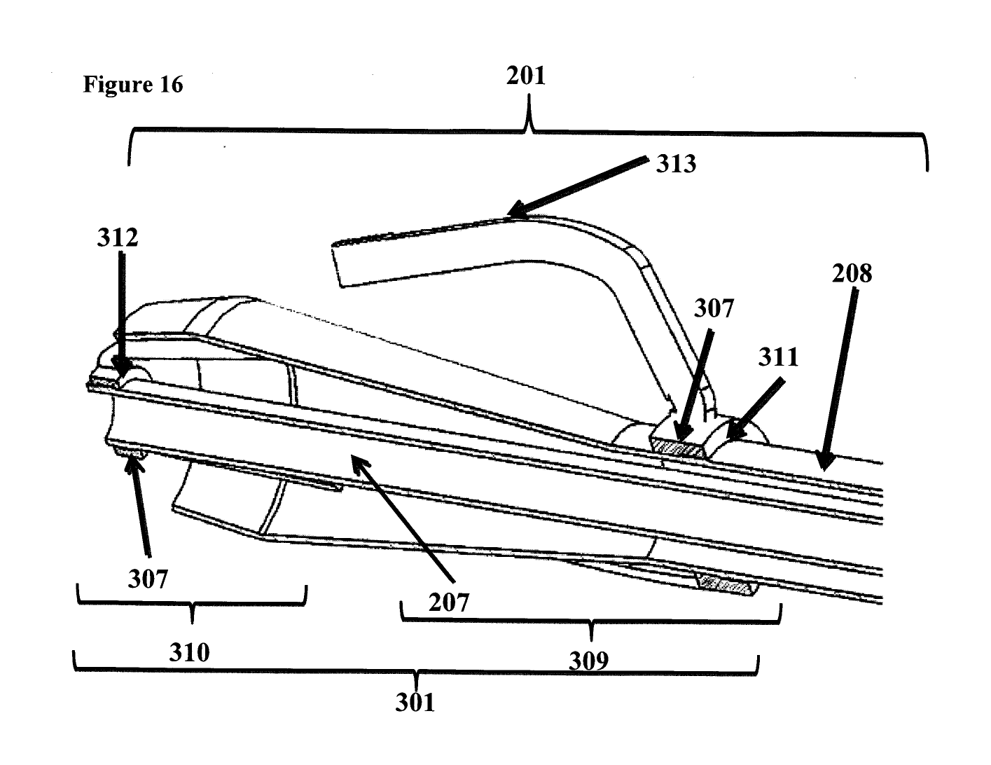

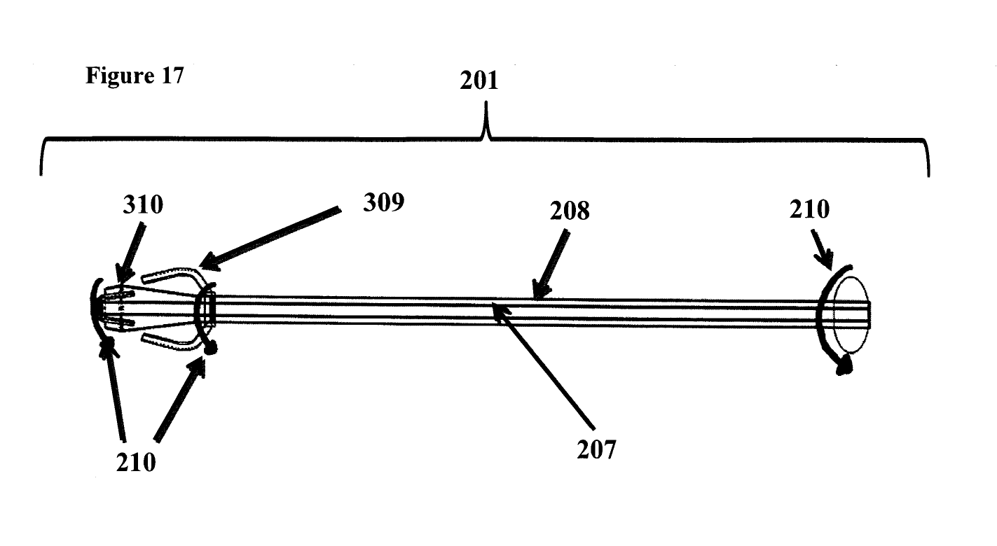

To release the expandable anastomosis structure (303) illustrated in FIGS. 9a-e, or other embodiments of the anastomosis structure, the proximal ring (311) and distal ring (312) are threaded (307), for example as illustrated in FIG. 16, to match corresponding threads on the inner catheter of the proximal portion of the catheter deployment system (208) and the inner catheter of the distal portion of the catheter deployment system (207). First the proximal grasper balloon (205) and the distal grasper balloon (203) are deflated allowing the proximal graspers (204) and distal graspers (202) to once again lie flat against the catheters (208, 207). The inner catheter of the proximal device (208) and the inner catheter of the distal device (207) are turned (210) as shown in FIG. 17 thus unscrewing them from the expandable anastomosis structure (303) proximal (311) and distal (312) rings that are threaded (307).

FIG. 16 shows a cross sectional view of the catheter deployment system (201) with the internal threads (307) between the proximal ring (311) and distal ring (312) exposed. The deployed expandable anastomosis structure (303) then remains in the vessel securing the anastomosis shown in FIG. 9f. Degradation of potential use of biodegradable materials will not occur until the anastomosis has acquired adequate strength, for example approximately 4 weeks. The degradation can be altered to match the specific anastomosis site.

Internal Flat Spring Release

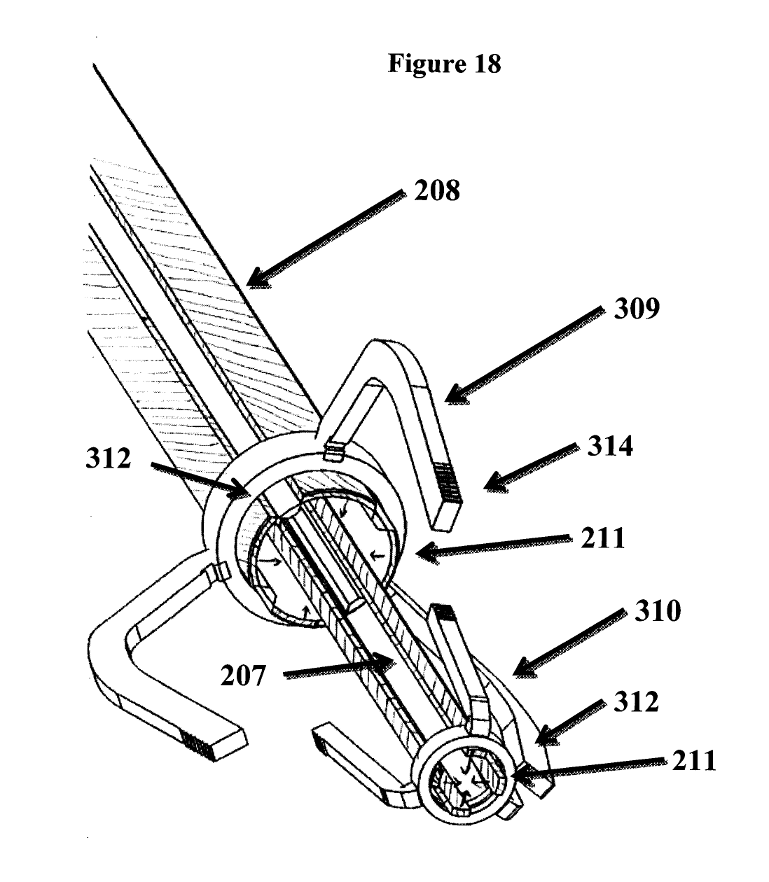

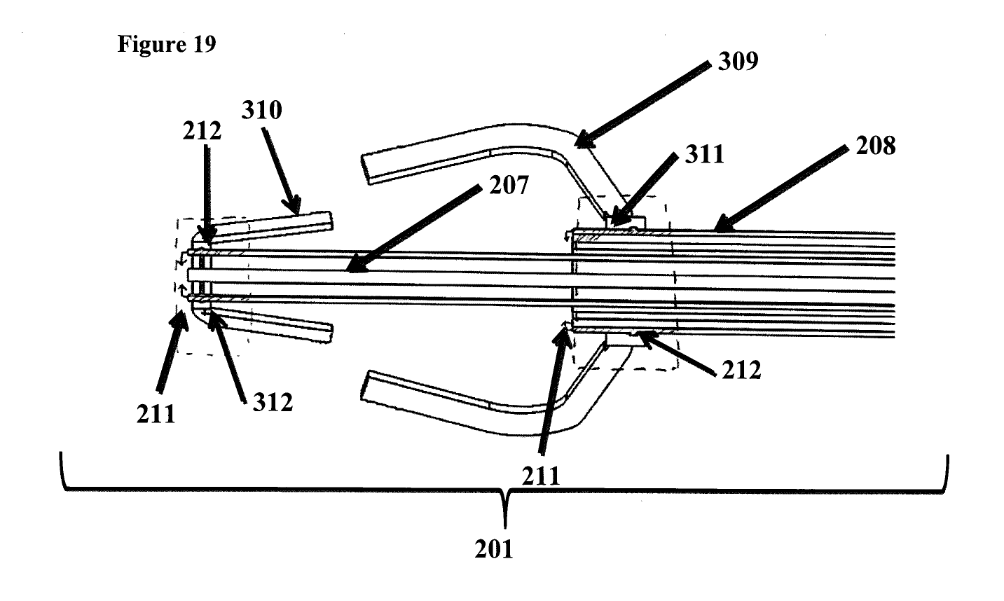

Another mechanism through which to release the expandable anastomosis structure (303), or other embodiments of the anastomosis structure, is through an internal flat spring as shown in FIGS. 18-19. The inner catheter of the proximal portion of the catheter deployment system (208) and inner catheter of the distal portion of the catheter deployment system (207) has a securement feature (212) as shown in FIG. 19 cross sectional area of the catheter deployment system (201). This securement feature (212) keeps the expandable anastomosis structure (303) on the catheter deployment system (201). The inner catheter of the proximal device (208) and the inner catheter of the distal device (207) can be squeezed (211) at the user end to pull a cable which will flex the sections of the catheters with securement features (212) inward. This releases the expandable anastomosis structure (303) from the catheter so that the deployment catheter system (201) can be removed through the proximal ring (311) and distal ring (312).

Expandable Anastomosis Structure Variations Deployment

The previously described method by which to deploy the expandable anastomosis structure (303) can be applied to the hourglass expandable anastomosis structure (302) and spiral expandable anastomosis structure (306). Additionally the barbed suture (304) feature to create longitudinal tension and the release mechanisms discussed (the screw release (307) and internal flat springe release with securement features (212)) are applicable to all other embodiments of the present disclosure.

Umbrella Anastomosis Structures

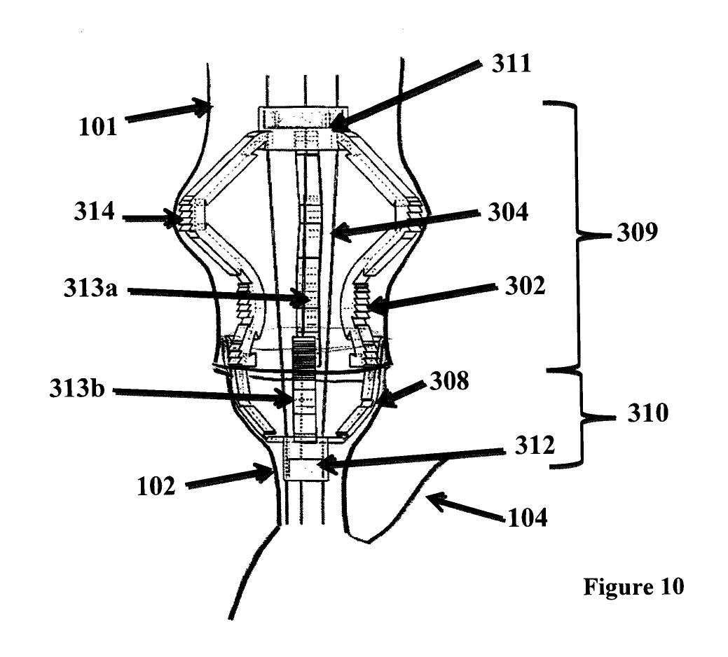

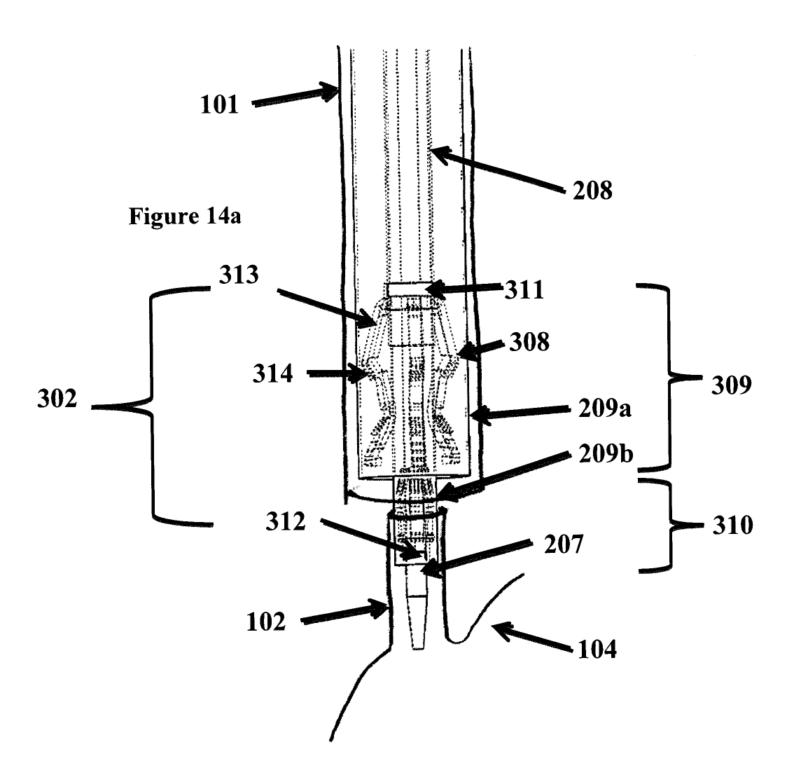

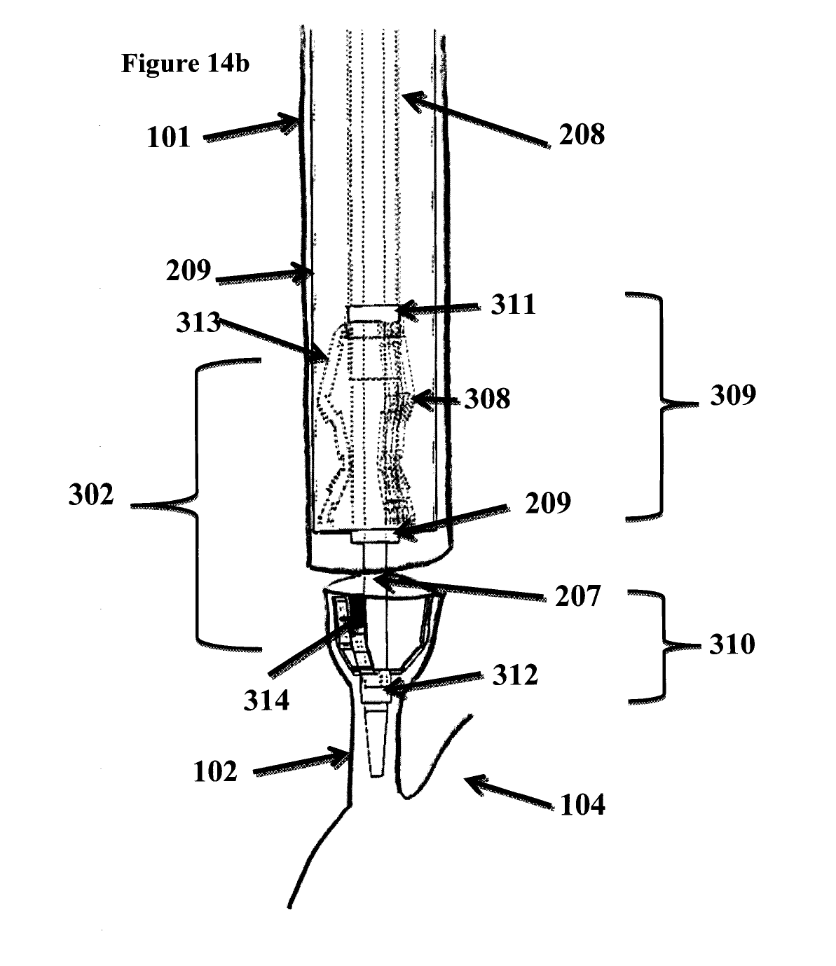

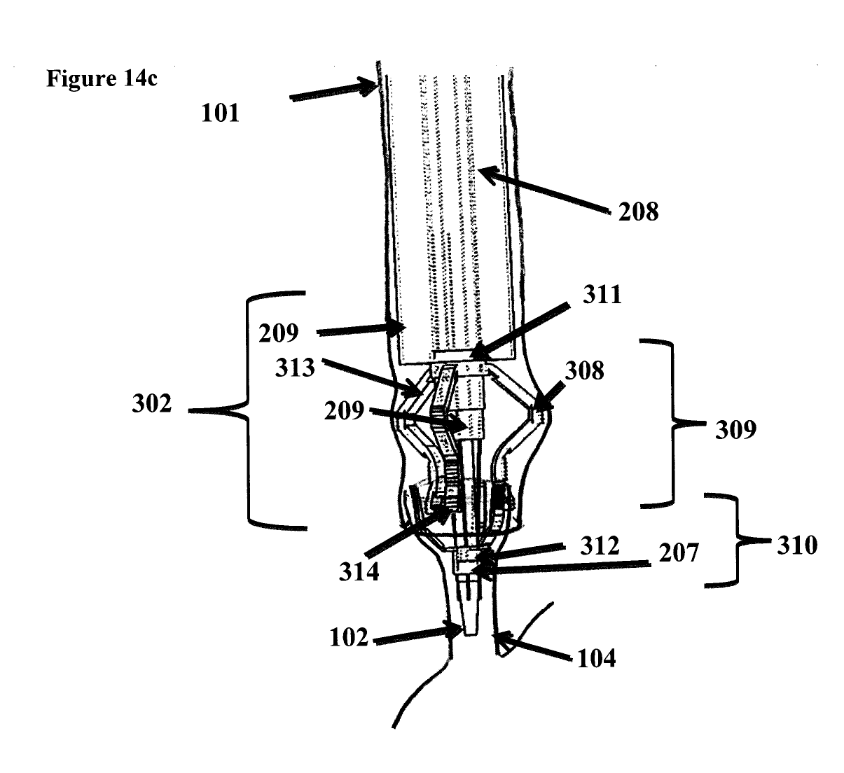

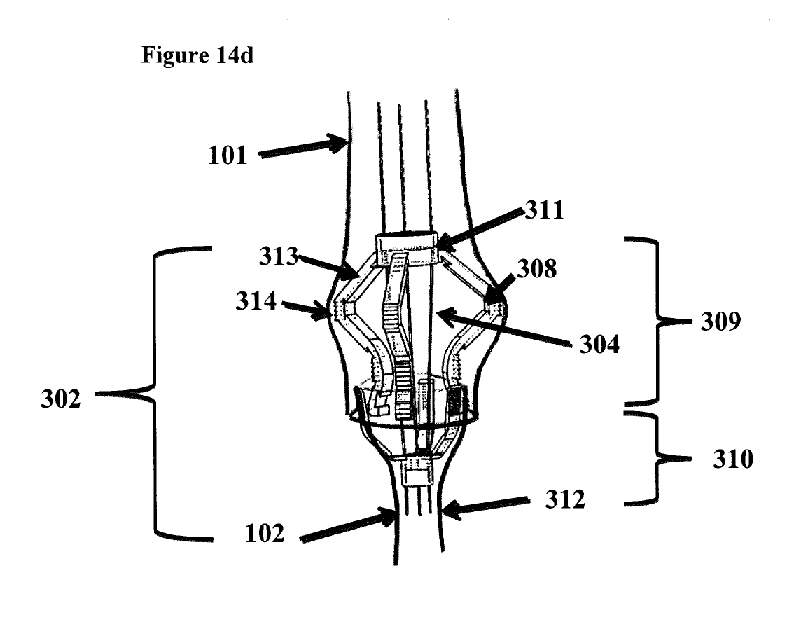

As described above, there are two categories of the anastomosis structure 301 shown in FIG. 3a-b. The second category is the hourglass-shaped umbrella anastomosis structure (302) which is shown in detail in FIG. 10. The hourglass-shaped umbrella anastomosis structure (302) includes two parts, a proximal anastomosis structure (309) and a distal anastomosis structure (310). The proximal anastomosis structure (309) of the hourglass-shaped umbrella anastomosis structure (302) has multiple arms (313a, 313b) containing multiple living hinges (308) set in the open state so that when the proximal anastomosis structure (309) is collapsed inside the anastomosis structure sheath (209), for example as illustrated in FIG. 14a, and then released, the proximal anastomosis structure (309) returns to the original shape as shown in FIG. 10.

In one embodiment, the hourglass-shaped umbrella anastomosis structure (302) has proximal anastomosis structure (309) and distal anastomosis structure (310) sections that are larger than the waist to account for the difference in size of the vessels to be anastomosed. In another embodiment, the sections are similar in size. The distal anastomosis structure (310) of the hourglass-shaped umbrella anastomosis structure (302) has multiple arms (313b) with multiple living hinges (308) to conform to the shape of the vessels. Similar to the proximal anastomosis structure (309), the distal anastomosis structure (310) is collapsed into the anastomosis structure sheath (209), for example as illustrated in FIG. 14a, and then released returning to the original shape as shown in FIG. 10 without permanent deformation due to collapsing into the sheath (209).

The hourglass-shaped umbrella anastomosis structure may contain multiple arms; the shown configuration has three proximal arms (313a) and three distal arms (313b). Other variations may have different numbers of arms (313a, 313b) on the two parts of the hourglass-shaped umbrella anastomosis structure (302). The arms may contain barbs (314), hooks or a microtexture that will engage and secure the hourglass-shaped umbrella anastomosis device (302) to the proximal vessel (101) and distal vessel (102) walls. In one embodiment, barbed sutures (304) run through the concentric proximal (311) and distal (312) rings to keep the two parts of the hourglass-shaped umbrella anastomosis structure (302) in longitudinal tension securing the anastomosis.

While barbed sutures (304) may be used to create the longitudinal tension between proximal and distal ends of the anastomosis structure, there are a number of mechanisms that may serve the same function such as but not limited to: mechanical locking features, clips to tie off the ends of the suture, a snap fit lock, or a material transition change such as the hardening of epoxy.

Rigid Distal Umbrella

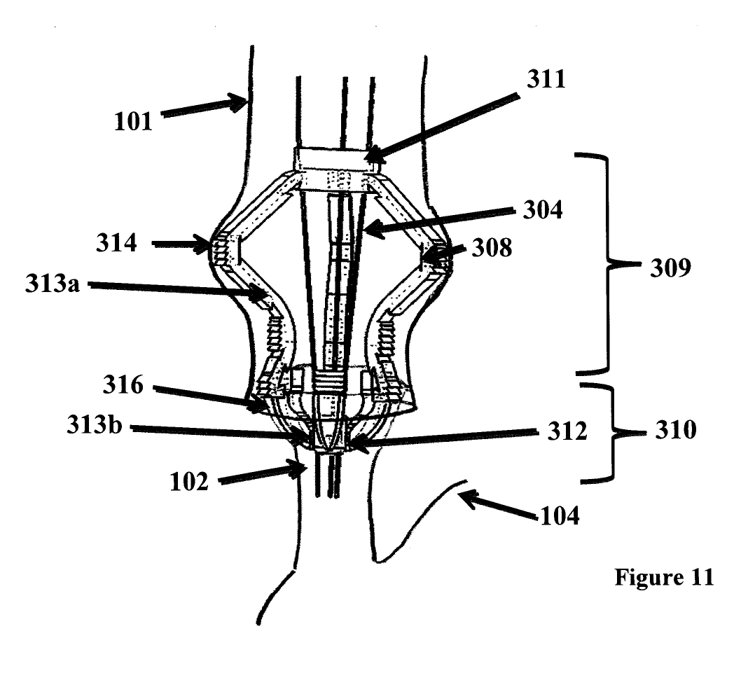

Another variation of the hourglass-shaped umbrella anastomosis structure (302) includes a configuration with a rigid distal umbrella anastomosis structure (316) as shown in FIG. 11. This rigid distal umbrella structure (316) is coupled with the proximal hourglass-shaped umbrella anastomosis structure (302), for example as illustrated in FIG. 10, to seal the anastomosis. The arms may contain barbs (314), hooks or a microtexture that will engage and secure the rigid distal umbrella anastomosis device (316) to the proximal vessel (101) and distal vessel (102) walls. The rigid distal umbrella anastomosis structure (316) would be oversized for the distal vessel (102) to maintain its position.

In one embodiment, barbed sutures (304) run through the distal ring (312) to keep the two parts of the hourglass-shaped umbrella (302) with rigid distal umbrella (316) anastomosis structure in longitudinal tension securing the anastomosis. The hourglass-shaped umbrella with rigid distal umbrella anastomosis structure (302) may contain multiple arms; the shown configuration in FIG. 11 has three proximal arms (313a) and three distal arms (313b). Other variations may have different numbers of arms (313a, 313b) on the two parts of the anastomosis structure (301).

While barbed sutures (304) may be used to create the longitudinal tension between proximal and distal ends of the anastomosis structure, there are a number of mechanism that may serve the same function such as but not limited to: mechanical locking features, clips to tie off the ends of the suture, a snap fit lock, or a material transition change such as the hardening of epoxy.

Flexible Arm Umbrella

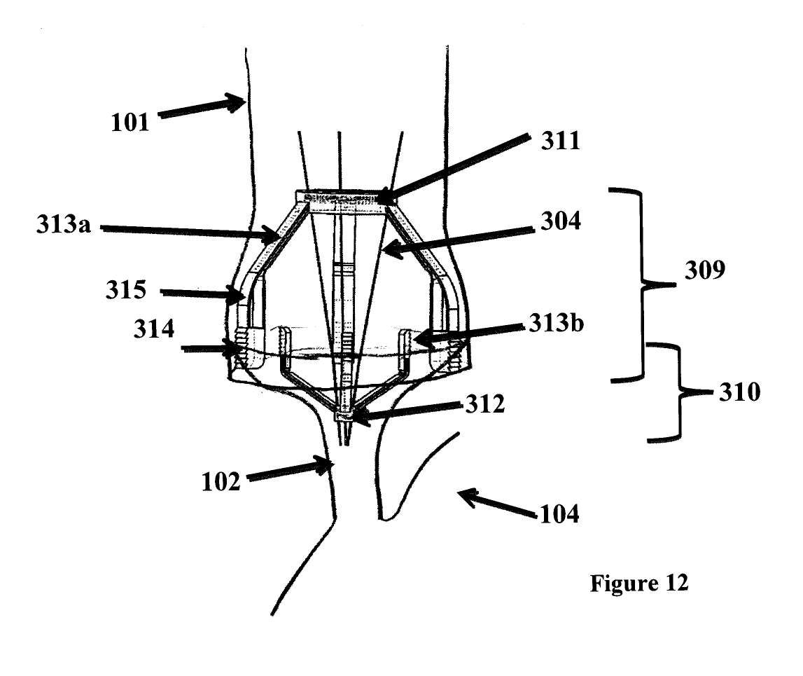

Another variation of the hourglass-shaped umbrella anastomosis structure (302) is the flexible arm umbrella anastomosis structure (315) shown in FIG. 12. This embodiment comprising of a proximal anastomosis structure (309) and a distal anastomosis structure (310) with the arms (313a, 313b) made from a flexible material that can withstand the deformation from being in the open or deployed state to the collapsed or sheathed state. The arms (313a, 313b) may contain barbs (314), hooks or a microtexture that will engage and secure the flexible arm umbrella anastomosis device (315) to the proximal vessel (101) and distal vessel (102) walls.

In one embodiment, barbed sutures (304) run through the proximal ring (311) and distal ring (312) to keep the two parts of the flexible arm umbrella anastomosis structure (315) in longitudinal tension securing the anastomosis. The flexible arm umbrella anastomosis structure (315) may contain multiple arms; the shown configuration in FIG. 12 has three proximal arms (313a) and three distal arms (313b). Other variations may have different numbers of arms (313a, 313b) on the two parts of the flexible arm umbrella anastomosis structure (315).

While barbed sutures (304) may be used to create the longitudinal tension between proximal and distal ends of the anastomosis structure, there are a number of mechanism that may serve the same function such as but not limited to: mechanical locking features, clips to tie off the ends of the suture, a snap fit lock, or a material transition change such as the hardening of epoxy.

Flat Spring Umbrella

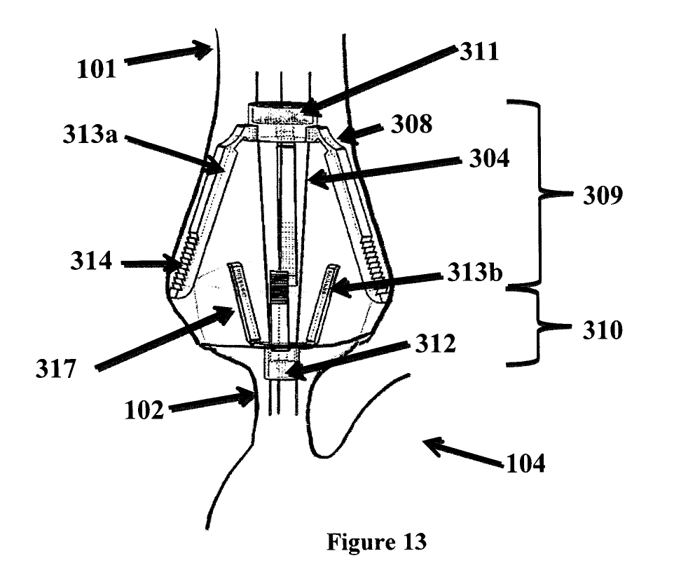

Another variation of the hourglass-shaped umbrella anastomosis structure (302) is the flat spring umbrella anastomosis structure (317) as shown in FIG. 13. This embodiment comprising of a proximal anastomosis structure (309) and distal anastomosis structure (310) with arms (313) made from a rigid material and containing one living hinge per arm (308). The living hinges (308) allow the proximal anastomosis structure (309) and distal anastomosis structure (310) to collapse into an anastomosis structure sheath (209) and then open to engage the vessel walls when unsheathed. The arms may contain barbs (314), hooks or a microtexture that will engage and secure the flat spring umbrella anastomosis device (317) to the proximal vessel (101) and distal vessel (102) walls.

In one embodiment, barbed sutures (304) run through the distal ring (312) and proximal ring (311) to keep the two parts of the flat spring umbrella anastomosis structure (317) in longitudinal tension securing the anastomosis. The flat spring umbrella anastomosis structure (317) may contain multiple arms; the shown configuration in FIG. 13 has three proximal arms (313a) and three distal arms (313b). Other variations may have different numbers of arms (313a, 313b) on the two parts of the flat spring umbrella anastomosis structure (317).

While barbed sutures (304) may be used to create the longitudinal tension between proximal and distal ends of the anastomosis structure, there are a number of mechanism that may serve the same function such as but not limited to: mechanical locking features, clips to tie off the ends of the suture, a snap fit lock, or a material transition change such as the hardening of epoxy.

All four embodiments; hourglass-shaped umbrella anastomosis structure (302), flexible arm anastomosis structure (315), hourglass-shaped umbrella with rigid distal umbrella anastomosis structure (316), and flat spring anastomosis structure (317), may be constructed of biodegradable materials or a combination of biodegradable and non-biodegradable materials such as but not limited to: biodegradable-(Polyglycolic acid (PGA), poly lactic-co-glycolic acid (PLGA), Poly-lactic acid (PLA), poly-L-lactide (PLLA), collagen, Magnesium Alloys, Iron based alloys, or any combination of the previously mentioned materials) or non-biodegradable-(stainless steel, Nitinol, cobalt alloys, and platinum alloys). An advantage of using biodegradable materials is that it allows for the anastomosis structure to be used more specifically in children without halting growth of the child resulting in strictures later in life.

Deployment of Umbrella Structure

FIGS. 14a-d show a method of deploying an hourglass-shaped umbrella anastomosis structure (302) into body vessels (101, 102) to create an anastomosis. FIG. 14a illustrates a first step of lowering the catheter deployment system 201 into a distal body vessel portion (102, e.g., distal esophagus portion). The proximal anastomosis structure (309) and distal anastomosis structure (310) are collapsed within anastomosis structure sheaths (209a, 209b), respectively, which lie on the concentric inner catheter of the proximal portion of the catheter deployment system (208) and inner catheter of the distal portion of the catheter deployment system (207). The collapsed hourglass-shaped umbrella anastomosis structure (302) is advanced into the distal most vessel (102) of the atresia site over the catheters as shown in FIG. 14a.

FIG. 14b illustrates a second step of releasing the distal anastomosis structure (310) from the anastomosis structure sheath (209b) and allowing it to engage the distal vessel (102) wall. The distal anastomosis structure (310) is unsheathed thus returning to the expanded state into the distal vessel (102) as shown in FIG. 14b. The living hinges (308) on the arms of the hourglass-shaped umbrella anastomosis structure (302) allow the structure to contour to the distal vessel (102) wall and the barbs (314) allow the distal anastomosis structure (310) to secure into the distal vessel wall (102). The inner catheter of the distal portion of the catheter deployment system (207) is pulled by the operator to move the distal vessel (102) with the distal anastomosis structure (310) into the proximal vessel (101) as shown in FIG. 14c.

As the distal anastomosis structure (310) is moved upward there are barbed sutures (304) passing through straight channels in the proximal ring (311) shown in FIG. 7 and maintaining a fixed position on the distal ring (312). FIG. 7 shows that the barbs (304) are angled in such a way that the suture (304) can pass up through the proximal ring (311) but not back through, so that the proximal ring (311) and distal ring (312) can move closer to each other but not further away.

FIG. 14c illustrates a third step of pulling the distal vessel 102 into the proximal vessel 101 and releasing the proximal anastomosis structure (309) from the sheath (209a) to allow it to expand and engage the proximal vessel (101) wall (e.g., esophageal wall). The proximal anastomosis structure (309) is unsheathed by pulling back the anastomosis structure sheath (209a). The proximal anastomosis structure (309) returns to the expanded state into the proximal vessel (101) wall as well as into the distal vessel (102) wall. The hourglass-shaped umbrella anastomosis structure (302) is designed so that the living hinges (308) allow for some flexibility of the structure to conform to the anastomosis site and the barbs (314), and allow the proximal anastomosis structure (310) to secure into the proximal vessel (101).

Other methods of creating the anastomosis after deploying the distal anastomosis structure (310) include removing the anastomosis sheath (209a) from the proximal anastomosis structure (309) and then actuating the inner catheter of the proximal portion of the catheter deployment system (208) and the inner catheter of the distal portion of the catheter deployment system (207) bringing the two structures together along with the proximal vessel (101) and distal vessel (102) walls. The barbed suture would lock the distance between the proximal anastomosis structure (309) and distal anastomosis structure (310) as the two are brought closer together.

FIG. 14d illustrates a fourth step of releasing both the proximal and distal anastomosis structures (309, 310) from the catheter deployment system (201) to leave them in place to seal the anastomosis. To release the hourglass-shaped umbrella anastomosis structure (302) shown in FIG. 14d, the proximal (311) and distal (312) rings are threaded (307), for example as illustrated in FIG. 16, to match corresponding threads on the inner catheter of the proximal portion of the catheter deployment system (208) and the inner catheter of the distal portion of the catheter deployment system (207). The inner catheter of the proximal portion of the catheter deployment system (208) and the inner catheter of the distal portion of the catheter deployment system (207) are turned (210) as shown in FIG. 17 thus unscrewing them from the hourglass-shaped umbrella anastomosis structure (302) proximal ring (311) and distal ring (312) that are threaded (307), for example as illustrated in FIG. 16.

FIG. 16 shows a cross sectional view of the catheter deployment system (201) with the internal threads (307) between the proximal ring (311) and distal ring (312) exposed. The deployed hourglass-shaped umbrella anastomosis structure (302) then remains in the vessel securing the anastomosis shown in FIG. 14d. The flexible arm umbrella anastomosis structure (315) and the flat spring anastomosis structure (317) are, in certain embodiments, deployed in the same fashion as the hourglass-shaped umbrella anastomosis structure (302). It should be noted that the hourglass-shaped umbrella anastomosis structure (302) can also be released using the internal flat spring release and securement feature (212), illustrated in FIG. 18 as discussed for the expandable anastomosis structure (303).

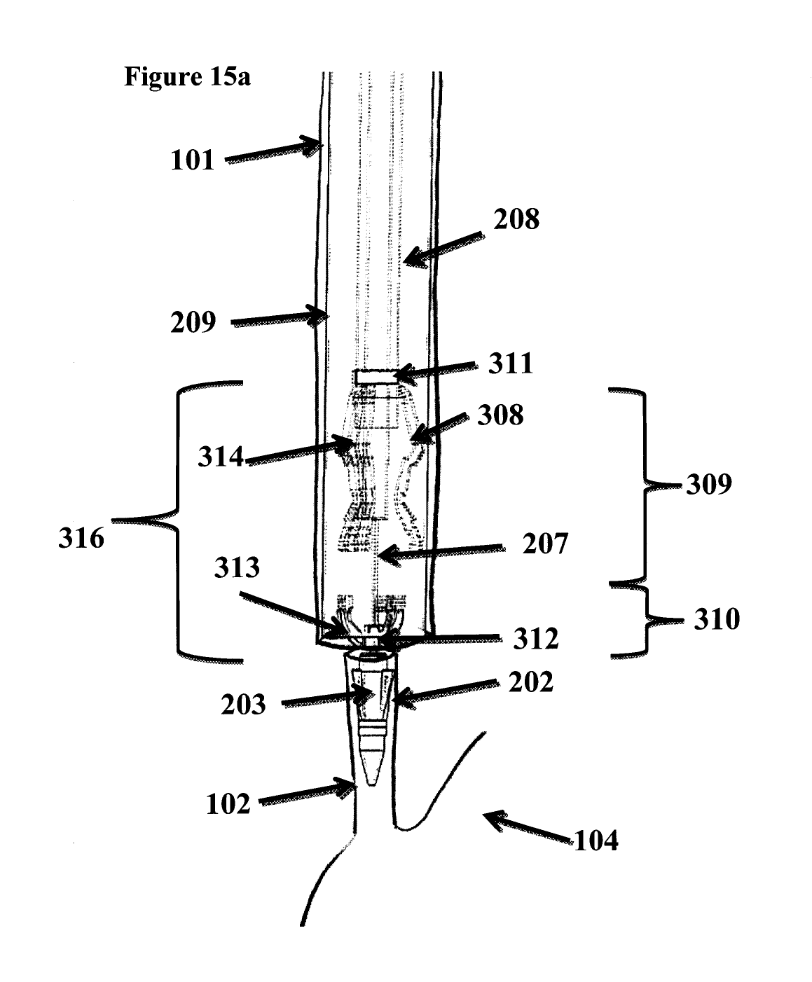

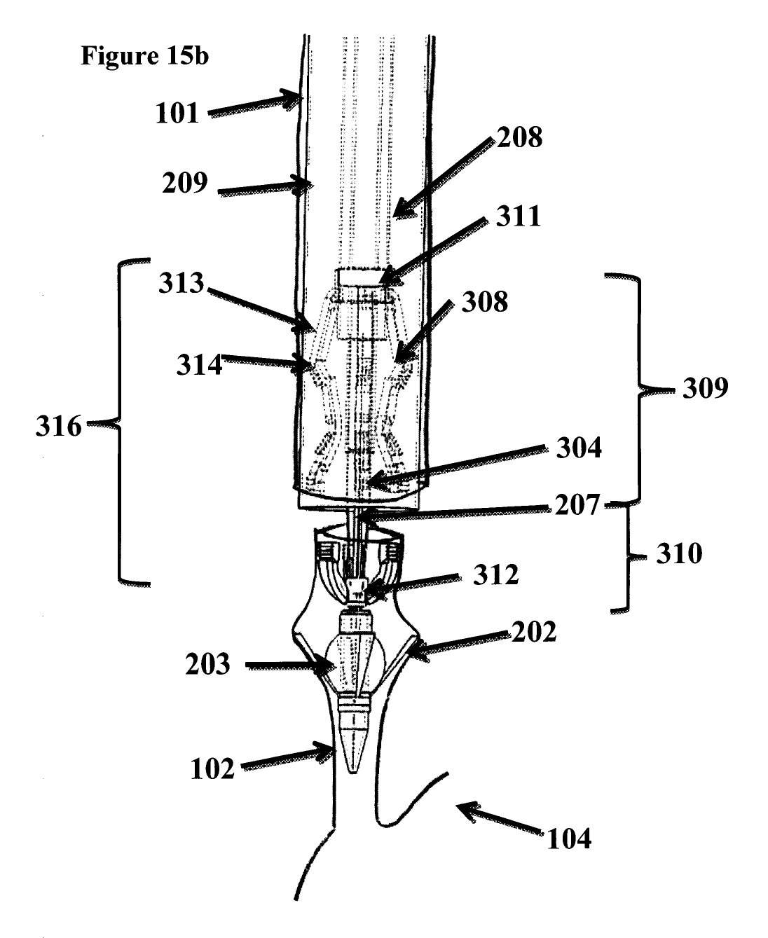

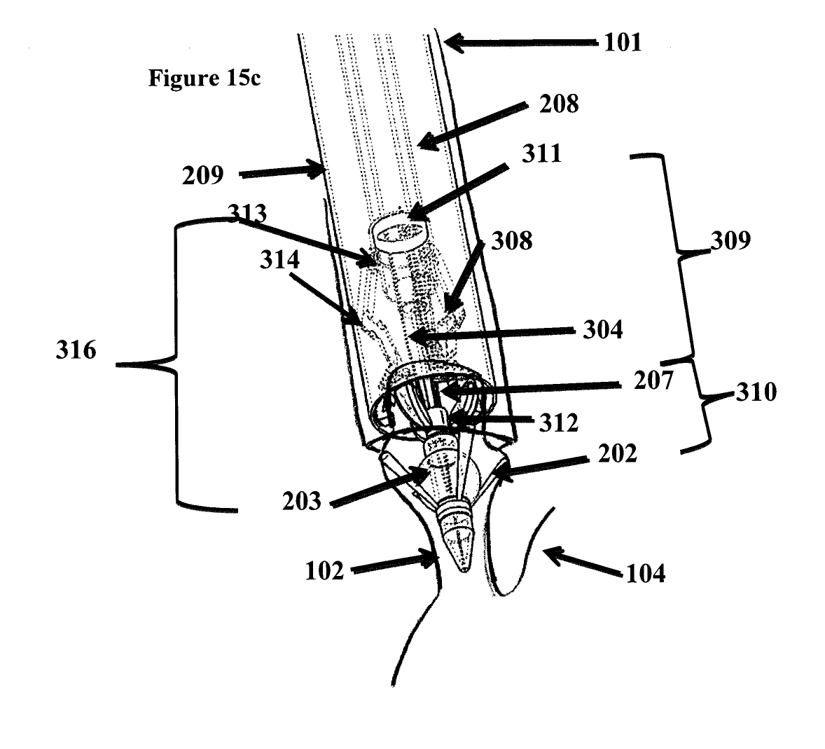

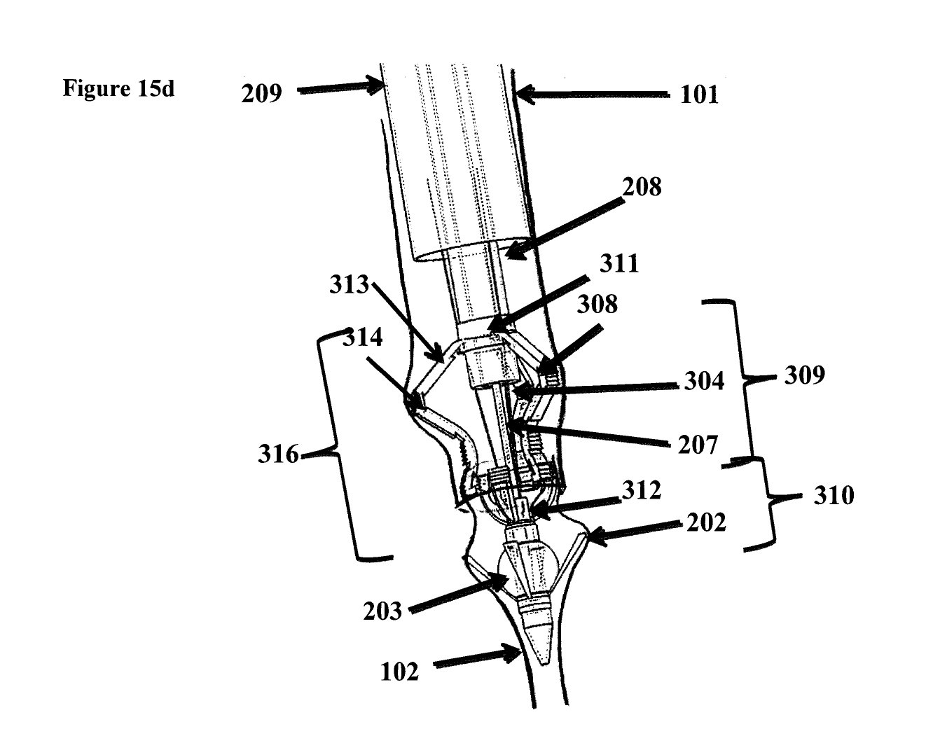

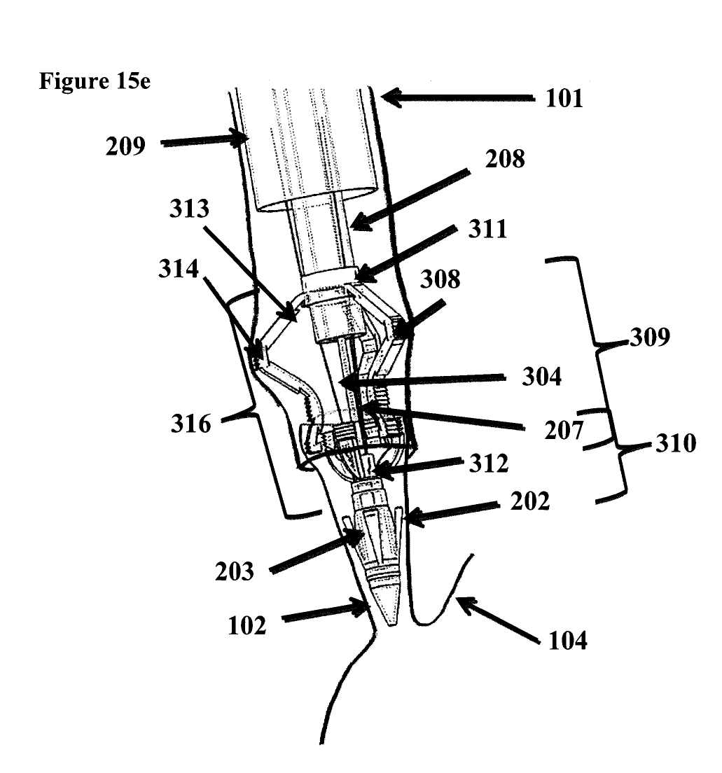

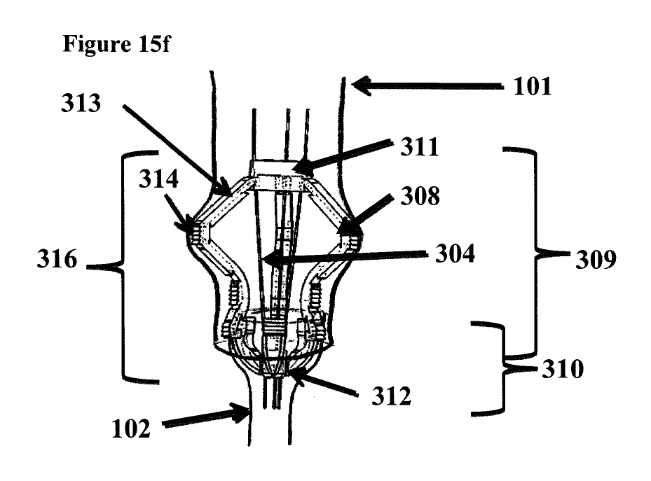

FIGS. 15a-f shows a method for deploying an exemplary hourglass-shaped umbrella anastomosis structure with a rigid distal umbrella anastomosis structure (316) into body vessels (101, 102), for example of an esophagus, to create an anastomosis.

FIG. 15a illustrates a first step in which the catheter deployment system (201) is lowered into the distal vessel (102). The hourglass-shaped umbrella with rigid distal umbrella anastomosis structure (316) is delivered to the atresia over concentric inner catheter of the proximal portion of the catheter deployment system (208) and inner catheter of the distal portion of the catheter deployment system (207) in the collapsed state as seen in FIG. 15a. The distal end of the catheter (207) is advanced through a natural orifice (e.g., the mouth) into the distal body vessel (102) (e.g., esophagus).

FIG. 15b illustrates a second step of inflating a distal grasper balloon (203) to engage distal graspers (202, e.g., distal esophagus graspers) with the distal vessel (102) wall. With the distal graspers (202) and distal grasper balloon (203) fully in the distal portion of the vessel (102), FIG. 15b shows the inflation of the distal graspers (202) that engage the distal vessel wall (102). The distal graspers (202) are made from Nitinol or a material of equivalent properties and may contain any number of arms; FIGS. 15a-f show four graspers.

The distal graspers (202) lay flat on the inner catheter of the distal portion of the catheter deployment system (207) when the grasper balloon (203) is deflated. The distal graspers (202) are attached to the inner catheter of the distal portion of the catheter deployment system (207) just below the distal grasper balloon (203) so that they can flex outward without permanent deformation and then again lay flat when the distal grasper balloon is deflated (203). With the distal vessel (102) secured with the distal graspers (202) the rigid distal umbrella anastomosis structure (316) can be placed in the distal vessel (102) and the barbs (314) can engage and secure the structure to the distal vessel (102) wall as shown in FIG. 15b.

The inner catheter of the distal portion of the catheter deployment system (207) can move independently of the inner catheter of the proximal portion of the catheter deployment system (208) and anastomosis structure sheath (209). FIG. 15c illustrates a fourth step in which the distal graspers (202) are used to pull the distal vessel (102) into the proximal vessel (101). With the distal vessel (102) wall engaged by the distal graspers (202), the distal vessel (102) is pulled into the proximal vessel (101) by moving the inner catheter of the distal portion of the catheter deployment system (207) and the distal graspers (202) independently of the inner catheter of the proximal portion of the catheter deployment system (208) so that there is an overlapping of the two proximal (101) and distal (102) vessel ends.

As the rigid distal umbrella anastomosis structure (316) is moved towards the proximal anastomosis structure (309) there are barbed sutures (304) passing through straight channels in the proximal ring (311) shown in FIG. 7 and maintaining a fixed position on the distal ring (312). FIG. 7 shows that the barbs (304) are angled in such a way that the suture (304) can pass up through the proximal ring (311) but not back through, so that the proximal ring (311) and distal ring (312) can move closer to each other but not further away.

FIG. 15d illustrates a fourth step in which the proximal portion of the anastomosis structure (309) is released from the sheath (209) to allow it to expand and engage the proximal and distal vessel (101, 102) walls. With the proximal vessel (101) and distal vessel (102) overlapped the proximal portion of the anastomosis structure (309) is unsheathed by sliding the anastomosis structure sheath (209) up, revealing the proximal anastomosis structure (309). FIG. 15d shows the proximal anastomosis structure (309) returning to the expanded state into the proximal vessel (101) wall as well as into the distal vessel (102) wall. The hourglass-shaped umbrella anastomosis structure (302) is designed so that the living hinges (308) allow for some flexibility of the structure to conform to the anastomosis site and the barbs (314) allow the proximal anastomosis structure (310) to secure into the proximal vessel (101).

FIG. 15e illustrates a fifth step in which the distal grasper balloon (203) is deflated to pull the distal graspers 202 from the distal vessel (102) wall. Prior to release of the hourglass-shaped umbrella with rigid distal umbrella anastomosis structure, the distal grasper balloon (203) is deflated, causing the distal graspers (202) to lie flat on the inner catheter of the distal portion of the catheter deployment system (207), as illustrated for example in FIG. 15e.

Other methods of creating the anastomosis after deploying the distal anastomosis structure (310) include removing the anastomosis sheath (209) from the proximal anastomosis structure (209) and then actuating the inner catheter of the proximal portion of the catheter deployment system (208) and the inner catheter of the distal portion of the catheter deployment system (207) bringing the two structures together along with the proximal vessel (101) and distal vessel (102) walls. The barbed suture would lock the distance between the proximal anastomosis structure (309) and distal anastomosis structure (310) as the two are brought closer together.

FIG. 15f illustrates a sixth step in which both the proximal and distal hourglass-shaped umbrella anastomosis structures (309, 310) are released from their respective catheters in the catheter deployment system (201) leaving them in place to seal the anastomosis. To release the hourglass-shaped umbrella with rigid distal umbrella anastomosis structure (316), as shown in FIG. 15f, the proximal (311) and distal (312) rings are threaded (307), for example as illustrated in FIG. 16, to match corresponding threads on the inner catheter of the proximal portion of the catheter deployment system (208) and the inner catheter of the distal portion of the catheter deployment system (207). The inner catheter of the proximal portion of the catheter deployment system (208) and the inner catheter of the distal portion of the catheter deployment system (207) are turned (210) as shown in FIG. 17 thus unscrewing them from the hourglass-shaped umbrella with rigid distal umbrella anastomosis structure (316) proximal (311) and distal (312) rings that are threaded (307).

FIG. 16 shows a cross sectional view of the catheter deployment system (201) with the internal threads (307) between the proximal ring (311) and distal ring (312) exposed. The deployed hourglass-shaped umbrella with rigid distal umbrella anastomosis structure (302) then remains in the body vessel securing the anastomosis shown in FIG. 15f. It should be noted that the hourglass-shaped umbrella with rigid distal umbrella anastomosis structure (315) can also be released using the internal flat spring release and securement feature (212), illustrated in FIG. 15 and as discussed for the expandable anastomosis structure (303).

Numerous modifications and variations of the embodiments of the present disclosure are possible in light of the above teachings. It is therefore to be understood that within the scope of the appended claims, the embodiments may be practiced otherwise than as specifically described herein.

1. An apparatus for performing anastomosis of distal and proximal vessels, the apparatus comprising:
a distal anastomosis section, including
a distal hub configured to detachably connect to a distal catheter, and
a distal engagement segment connected to the distal hub and including one or more distal arm segments that are configured to engage an inner wall of the distal vessel.

2. The apparatus according to claim 1, further comprising:
the distal catheter including one or more distal graspers that are configured to engage the inner wall of the distal vessel, the distal catheter being configured to pull the distal vessel towards the proximal vessel while the one or more distal graspers engage the inner wall of the distal vessel.

3. The apparatus according to claim 1, further comprising:
a proximal anastomosis section, including
a proximal hub configured to detachably connect to a proximal catheter, and
a proximal engagement segment connected to the proximal hub and including one or more proximal arm segments that are configured to engage the proximal vessel.

4. The apparatus according to claim 3, wherein
the distal anastomosis section and the proximal anastomosis section are part of a single anastomosis structure, and
the distal engagement segment is connected to the proximal engagement segment.

5. The apparatus according to claim 4, wherein the single anastomosis structure is an hourglass shaped structure.

6. The apparatus according to claim 4, wherein one or a combination of the one or more distal arm segments and the one or more proximal arm segments contain living hinges.

7. The apparatus according to claim 4, further comprising:
a fluid impermeable membrane configured to prevent fluid leakage through the distal and proximal engagement segments.

8. The apparatus according to claim 4, further comprising:
the distal catheter including one or more distal graspers that are configured to engage the inner wall of the distal vessel, the distal catheter being configured to pull the distal vessel towards the proximal vessel while the one or more distal graspers engage the inner wall of the distal vessel, and
the proximal catheter including on or more proximal graspers that are configured to engage the inner wall of the proximal vessel to stabilize the proximal vessel while the distal vessel is being pulled towards the proximal vessel.

9. The apparatus according to claim 8, wherein
the distal engagement segment is configured to engage the distal vessel and the proximal engagement segment is configured to engage the proximal vessel when the distal and proximal catheters are moved towards each other.

10. The apparatus according to claim 3, wherein
the distal and proximal anastomosis sections are separate anastomosis structures.

11. The apparatus according to claim 10, wherein the distal anastomosis section further includes a distal deployment mechanism configured to deploy the distal engagement segment such that the one or more distal arm segments engage the inner wall of the distal vessel.

12. The apparatus according to claim 11, wherein
the distal deployment mechanism includes a sheath that compresses the one or more distal arm segments against the distal catheter, and
the one or more distal arm segments are configured to deploy when the sheath is removed from the one or more distal arm segments.

13. The apparatus according to claim 11, further comprising:
the distal catheter configured to pull the distal vessel towards the proximal vessel while the one or more distal arm segments engage the inner wall of the distal vessel.

14. The apparatus according to claim 10, wherein the distal anastomosis section is a rigid structure.

15. The apparatus according to claim 3, wherein one or a combination of the distal and proximal anastomosis sections is a flexible structure that is configured to expand radially away from the distal catheter.

16. The apparatus according to claim 14, further comprising:
the distal catheter including one or more distal graspers that are configured to engage the inner wall of the distal vessel, the distal catheter being configured to pull the distal vessel towards the proximal vessel while the one or more distal graspers engage the inner wall of the distal vessel.

17. The apparatus according to claim 10, wherein the proximal anastomosis section further includes a proximal deployment mechanism configured to deploy the proximal engagement segment such that the one or more proximal arm segments engage the inner wall of the proximal vessel.

18. The apparatus according to claim 10, further comprising:
a connector configured to couple the distal engagement segment to the proximal engagement segment.

19. The apparatus according to claim 18, wherein the connector is a barbed suture.

20. A method for performing anastomosis of distal and proximal vessels, the method comprising:
inserting a distal catheter through a proximal vessel and into a distal vessel, the distal catheter having detachably mounted thereon a distal anastomosis section, including
a distal hub configured to detachably connect to the distal catheter, and
a distal engagement segment connected to the distal hub and including one or more distal arm segments that are configured to engage an inner wall of the distal vessel.

21. The method according to claim 20, wherein the step of inserting comprises:
Inserting the distal catheter through the proximal vessel via a natural orifice.

22. The method according to claim 20, further comprising:
deploying one or more distal graspers that are connected to the distal catheter and configured to engage the inner wall of the distal vessel; and
retracting the distal catheter to pull the distal vessel towards the proximal vessel while the one or more distal graspers engage the inner wall of the distal vessel.

23. The method according to claim 20, further comprising:
inserting a proximal catheter through the proximal vessel, the proximal catheter having detachably mounted thereon a proximal anastomosis section, including
a proximal hub, and
a proximal engagement segment connected to the proximal hub and including one more proximal arm segments that are configured to engage the proximal vessel.

24. The method according to claim 23, wherein
the distal anastomosis section and the proximal anastomosis section are part of a single anastomosis structure, and
the distal engagement segment is connected to the proximal engagement segment.

25. The method according to claim 24, further comprising:
deploying one or more distal graspers that are connected to the distal catheter and configured to engage the inner wall of the distal vessel;
deploying one or more proximal graspers that are connected to the proximal catheter and configured to engage the inner wall of the proximal vessel to stabilize the proximal vessel while the distal vessel is being pulled towards the proximal vessel; and
retracting the distal catheter to pull the distal vessel towards the proximal vessel while the one or more distal graspers engage the inner wall of the distal vessel.

26. The method according to claim 25, further comprising:
moving the distal and proximal catheters towards each other such that the distal engagement segment engages the distal vessel and the proximal engagement segment engages the proximal vessel.

27. The method according to claim 23, wherein
the distal and proximal anastomosis sections are separate anastomosis structures.

28. The method according to claim 27, further comprising:
actuating a distal deployment mechanism, included in the distal anastomosis section, to deploy the distal engagement segment such that the one or more distal arm segments engage the inner wall of the distal vessel.

29. The method according to claim 28, further comprising:
retracting the distal catheter to pull the distal vessel towards the proximal vessel while the one or more distal arm segments engage the inner wall of the distal vessel.

30. The method according to claim 27, wherein the distal anastomosis section is a rigid structure.

31. The method according to claim 30, further comprising:
deploying one or more distal graspers that are connected to the distal catheter and configured to engage the inner wall of the distal vessel; and
retracting the distal catheter to pull the distal vessel towards the proximal vessel while the one or more distal graspers engage the inner wall of the distal vessel.

32. The method according to claim 27, further comprising:
actuating a proximal deployment mechanism, included in the proximal anastomosis section, to deploy the proximal engagement segment such that the one or more proximal arm segments engage the inner wall of the proximal vessel.
